(19) United States
(12) Patent Application Publication
(10) Pub. No.: US 2024/0050477 A1
(43) Pub. Date: Feb. 15, 2024

(54) LIPID NANOPARTICLE

(71) Applicant: NATIONAL UNIVERSITY CORPORATION HOKKAIDO UNIVERSITY, Sapporo-shi, Hokkaido (JP)

(72) Inventors: Takashi Nakamura, Sapporo-shi, Hokkaido (JP); Hideyoshi Harashima, Sapporo-shi, Hokkaido (JP); Yusuke Sato, Sapporo-shi, Hokkaido (JP); Koharu Yamada, Sapporo-shi, Hokkaido (JP); Taisei Nakade, Sapporo-shi, Hokkaido (JP)

(73) Assignee: NATIONAL UNIVERSITY CORPORATION HOKKAIDO UNIVERSITY, Sapporo-shi, Hokkaido (JP)

(21) Appl. No.: 18/277,409

(22) PCT Filed: Feb. 17, 2022
(86) PCT No.: PCT/JP2022/006427

§ 371 Date: Aug. 16, 2023

(30) Foreign Application Priority Data
Feb. 17, 2021 (JP) 2021-023184

(51) Int. Cl. A61K 35/17; C12N 5/0783; A61K 9/127; C12N 15/113; A61K 31/7105; A61P 35/00

(52) U.S. Cl. CPC A61K 35/17; C12N 5/0646; A61K 9/127; C12N 15/113; A61K 31/7105; A61P 35/00

(57) ABSTRACT

The present invention provides a lipid nanoparticle and the like containing a pH-sensitive cationic lipid represented by formula (1) (in formula (1), R1 and R2 are each independently a straight-chain C10-14 alkyl group, a straight-chain C10-20 alkenyl group having one or two unsaturated bonds, or —CH(R5)(R6)(where R5 and R6 are each independently a straight-chain C5-10 alkyl group); p represents an integer of 3-8; and R3 and R4 are each independently a methyl group or an ethyl group].

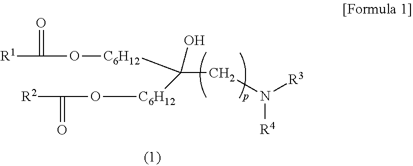

### TECHNICAL FIELD

The present invention relates to a lipid nanoparticle useful as a gene delivery carrier capable of delivering natural killer (NK) cells with high efficiency.

### BACKGROUND ART

Lipid nanoparticles (LNPs) are utilized as carriers for encapsulating lipid-soluble drugs or nucleic acids such as siRNA (small interfering RNA) or mRNA and delivering them to target cells. For example, a lipid nanoparticle containing, as a constituent lipid, a pH-sensitive cationic lipid which is electrically neutral at physiological pH and turns into cationic property in a weakly acidic pH environment such as endosome has been reported as a lipid nanoparticle serving as a carrier for efficiently delivering a nucleic acid such as siRNA into target cells (Patent Literature 1).

Chimeric antigen receptor-T cell therapy (CAR-T therapy) exhibits marked therapeutic effects on blood cancer and largely contributes to cancer treatment. However, this therapy has plenty of challenges such as safety of gene engineering using virus carriers, complicated production, maintenance of the quality of T cells, and high cost. NK cell therapy is expected to follow cell therapy using T cells (Non Patent Literature 1). NK cells are effector cells that are effective for cancers having a mutation so as to avoid attack from T cells. Since the NK cells in blood are fewer than T cells and are therefore difficult to secure at a sufficient number, clinical trials using a human NK cell line NK-92 are ongoing. Use of such a cell line has many advantages that a sufficient number of cells can be secured, the cells are also preservable, and quality is easily kept constant, for example, (Non Patent Literature 2). Hence, future advancement of NK cell therapy using a human NK cell line such as NK-92 is expected.

Gene engineering or gene expression control is an important technique of enhancing the functions of NK cells. For efficient enhancement, it is necessary to use virus carriers. Therefore, this technique still has challenges in terms of safety or cost. The present applicant et al. have successfully developed a lipid nanoparticle (LNP) capable of efficiently introducing siRNA to human immunocyte lines (Non Patent Literature 3). This lipid nanoparticle has a pH-sensitive cationic lipid YSK12-C4 as a constituent lipid and is therefore efficiently taken up into human cells such as lymphocytes or monocytes.

### CITATION LIST

### Patent Literature

Patent Literature 1: International Publication No. WO 2018/230710
Patent Literature 2: International Publication No. WO 2018/190423

### Non Patent Literature

Non Patent Literature 1: Shimasaki et al., Nature Reviews Drug Discovery, 2020, vol. 19, p. 200-218
Non Patent Literature 2: Klingemann et al., Frontiers Immunology, 2016, vol. 7, Article 91
Non Patent Literature 3: Nakamura et al., Scientific Reports, 2016, 6: 37849
Non Patent Literature 4: Nakamura et al., Molecular Pharmaceutics, 2018, vol. 15, p. 2142-2150
Non Patent Literature 5: Sato et al., Journal of Controlled Release, 2019, vol. 295, p. 140-152
Non Patent Literature 6: Leung et al., Journal of Physical Chemistry C Nanomater Interfaces, 2012, vol. 116 (34), p. 18440-18450

### SUMMARY OF INVENTION

### Technical Problem

The lipid nanoparticle containing YSK12-C4 is a first-ever non-viral carrier that was able to efficiently introduce siRNA even to NK-92 cells, as compared with commercially available reagents. However, problems thereof are that: delivery efficiency to NK cells is not high in comparison with other immunocytes; and in addition, toxicity is found (Non Patent Literature 3). Although the toxicity was alleviated by decreasing the content of YSK12-C4 in the lipid nanoparticle, gene knockdown activity cannot be increased (Non Patent Literature 4).

An object of the present invention is to provide a lipid nanoparticle serving as a gene delivery carrier capable of delivery to NK cells with high efficiency.

### Solution to Problem

The present inventors have completed the present invention by finding that a pH-sensitive cationic lipid that has pKa on the order of 8.0 to 8.5 and has a hydrocarbon chain having a particular structure containing an ester structure is contained as a constituent lipid in a lipid nanoparticle in which a nucleic acid is encapsulated, whereby excellent nucleic acid introduction efficiency to human NK cell lines and low toxicity can be achieved.

Specifically, the present invention provides the following lipid nanoparticle and the like.

[1] A lipid nanoparticle comprising a pH-sensitive cationic lipid represented by the following formula (1):

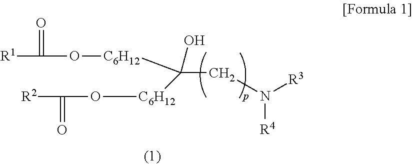

wherein R¹ and R² are each independently a straight-chain C₁₀₋₁₄ alkyl group, a straight-chain C₁₀₋₂₀ alkenyl group having one or two unsaturated bonds, or —CH(R⁵)(R⁶), where R⁵ and R⁶ are each independently a straight-chain C₅₋₁₀ alkyl group; p represents an integer of 3-8; and R³ and R⁴ are each independently a methyl group or an ethyl group.

[2] The lipid nanoparticle according to [1], further comprising sterol and a polyalkylene glycol-modified lipid.

[3] The lipid nanoparticle according to [1] or [2], wherein a ratio of an amount of the pH-sensitive cationic lipid to the total amount of the lipids constituting the lipid nanoparticle is 20 mol % or more.

[4] The lipid nanoparticle according to any of [1] to [3], comprising a nucleic acid.

[5] The lipid nanoparticle according to [4], wherein the nucleic acid is siRNA or mRNA.

[6] The lipid nanoparticle according to [4], wherein the nucleic acid is plasmid DNA.

[7] The lipid nanoparticle according to any of [4] to [6], wherein the nucleic acid is a gene to be expressed in an NK cell, or a functional nucleic acid that controls gene expression in an NK cell.

[8] An NK cell transfected with the lipid nanoparticle according to any of [1] to [7].

[9] A pharmaceutical composition comprising the lipid nanoparticle according to any of [1] to [7] or the NK cell according to [8] as an active ingredient.

[10] The pharmaceutical composition according to [9] for use in gene therapy.

[11] The pharmaceutical composition according to [9] for use in cancer treatment.

[12] The pharmaceutical composition according to for use in cancer immunotherapy.

[13] A kit for transforming an NK cell, comprising the lipid nanoparticle according to any of [1] to [7] and an NK cell.

[14] A method for transforming an NK cell, comprising introducing the lipid nanoparticle according to [7] to the NK cell so that the NK cell is transformed with the nucleic acid contained in the lipid nanoparticle.

[15] A method for suppressing a cancer, comprising administering a transformed NK cell obtained by the method for transforming an NK cell according to to an animal having a cancer tissue to reduce a size of the cancer tissue or to suppress increase in size of the cancer tissue.

[16] A method for expressing a gene or a functional nucleic acid, comprising administering the lipid nanoparticle according to [7] to a test animal so that the gene or the functional nucleic acid contained in the lipid nanoparticle is expressed in an NK cell of the test animal.

### Advantageous Effects of Invention

The lipid nanoparticle according to the present invention enables an encapsulated gene to be highly expressed in NK cells and also has low toxicity. Therefore, this lipid nanoparticle is useful as a NK cell-specific gene delivery carrier for use in gene therapy.

### BRIEF DESCRIPTION OF DRAWINGS

FIG. 1 is a diagram showing results of measuring the GAPDH gene knockdown activity of NK-92 cells transfected with each siGAPDH-encapsulated lipid nanoparticle in Example 1.

FIG. 2 is a diagram showing results of measuring the survival rate (%) of NK-92 cells transfected with each siGAPDH-encapsulated lipid nanoparticle in Example 1.

FIG. 3 is a diagram showing results of measuring the GAPDH gene knockdown activity of NK-92 cells transfected with each siGAPDH-encapsulated lipid nanoparticle (CL1H6-LNP) in Example 2.

FIG. 4 is a diagram showing results of measuring the survival rate (%) of NK-92 cells transfected with each siGAPDH-encapsulated lipid nanoparticle (CL1H6-LNP) in Example 2.

FIG. 5 is a diagram showing results of measuring the GAPDH gene knockdown activity of NK-92 cells transfected with each siGAPDH-encapsulated lipid nanoparticle (YSK12-LNP) in Example 2.

FIG. 6 is a diagram showing results of measuring the survival rate (%) of NK-92 cells transfected with each siGAPDH-encapsulated lipid nanoparticle (YSK12-LNP) in Example 2.

FIG. 7 is a diagram in which the gene knockdown activity and cell survival rate of YSK12-LNP (85%) (FIG. 7(A)) and CL1H6-LNP (25%) (FIG. 7(B)) were plotted with an siRNA concentration at the time of transfection on the abscissa and gene knockdown activity and a cell survival rate (%) on the ordinate in Example 2.

FIG. 8 is a diagram showing the half maximal effective concentration (EC₅₀) (nM) (FIG. 8(A)) and lethal median temperature (LC₅₀) (nM) (FIG. 8(B)) of YSK12-LNP (25%) and CL1H6-LNP (25%) in Example 2.

FIG. 9 is a diagram showing the gene knockdown activity (%) at a cell survival rate of 80% of YSK12-LNP (85%), YSK12-LNP (25%), and CL1H6-LNP (25%) in Example 2.

FIG. 10 is a diagram of a plot with the content ratio (% by mol) of YSK12-C4 or CL1H6 on the abscissa and EC₅₀ (nM) on the ordinate in Example 2.

FIG. 11 is a diagram showing results of measuring the GAPDH gene knockdown activity of NK-92 cells transfected with each siGAPDH-encapsulated lipid nanoparticle in Example 3.

FIG. 12 is a diagram showing results of measuring the survival rate (%) of NK-92 cells transfected with each siGAPDH-encapsulated lipid nanoparticle in Example 3.

FIG. 13 is a diagram showing results of measuring the GAPDH gene knockdown activity of KHYG-1 cells transfected with each siGAPDH-encapsulated lipid nanoparticle in Example 3.

FIG. 14 is a diagram showing results of measuring the survival rate (%) of KHYG-1 cells transfected with each siGAPDH-encapsulated lipid nanoparticle in Example 3.

FIG. 15 is a diagram showing results of measuring the gene knockdown activity of NK-92MI cells transfected with siGAPDH- or siSMAD3-encapsulated CL1H6-LNP in Example 4.

FIG. 16 is a diagram showing results of measuring the survival rate (%) of NK-92MI cells transfected with siGAPDH- or siSMAD3-encapsulated CL1H6-LNP in Example 4.

FIG. 17 is a diagram showing results of measuring time-dependent change in tumor volume of a mouse given PBS, untreated NK-92MI cells, or SMAD3-silenced NK-92MI cells in Example 5.

FIG. 18 is a diagram showing results of measuring the luciferase activity after 24 hours of NK-92 cells transfected with each Luc mRNA-encapsulated lipid nanoparticle in Example 6.

FIG. 19 is a diagram showing results of measuring the survival rate (%) of NK-92 cells transfected with each Luc mRNA-encapsulated lipid nanoparticle in Example 6.

FIG. 20 is a diagram showing results of measuring the luciferase activity after 24 hours of NK-92MI cells transfected with each Luc mRNA-encapsulated lipid nanoparticle in Example 7.

FIG. 21 is a histogram showing results of measuring the GFP expression after 24 hours of NK-92MI cells transfected with each GFP mRNA-encapsulated lipid nanoparticle in Example 8.

FIG. 22 is a diagram showing results (median value of fluorescence intensity) of measuring the GFP expression after 24 hours of NK-92MI cells transfected with each GFP mRNA-encapsulated lipid nanoparticle in Example 8.

FIG. 23 is a fluorescence microphotograph showing the GFP expression after 24 hours of NK-92 cells transfected with each GFP mRNA-encapsulated lipid nanoparticle in Example 8.

FIG. 24 is a diagram showing results of measuring the survival rate (%) of NK-92MI cells transfected with each GFP mRNA-encapsulated lipid nanoparticle in Example 8.

### DESCRIPTION OF EMBODIMENTS

Hereinafter, the mode for carrying out the present invention will be specifically described. In the specification of the present application, the term “X1 to X2, where X1 and X2 are real numbers that satisfy X1<X2” means “X1 or more and X2 or less”.

The lipid nanoparticle according to the present invention is a lipid nanoparticle containing a pH-sensitive cationic lipid represented by the general formula (1) given below (hereinafter, also referred to as the “the pH-sensitive cationic lipid of the present invention”). The lipid nanoparticle according to the present invention having the pH-sensitive cationic lipid represented by the general formula (1) as a constituent lipid has favorable delivery efficiency to NK cells and has toxicity kept low.

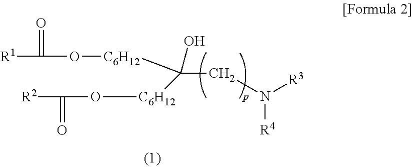

In the general formula (1), p represents an integer of 3-8 and is preferably 4.

In the general formula (1), R³ and R⁴ are each independently a methyl group or an ethyl group. Specifically, both R³ and R⁴ may be methyl groups; any one of R³ and R⁴ may be a methyl group, and the other may be an ethyl group; or both R³ and R⁴ may be ethyl groups. When a tertiary amino group assumes this structure, the pKa of the pH-sensitive cationic lipid represented by the general formula (1) can be on the order of 8.0 to 9.0, preferably on the order of 8.0 to 8.5, more preferably on the order of 8.0 to 8.3. When the pKa falls within this range, the lipid nanoparticle containing the pH-sensitive cationic lipid represented by the general formula (1) as a constituent lipid is favorably taken up into NK cells. For example, the pKa of the pH-sensitive cationic lipid of the general formula (1) wherein both R³ and R⁴ are methyl groups is on the order of 8.20; the pKa of the pH-sensitive cationic lipid of the general formula (1) wherein any one of R³ and R⁴ is a methyl group, and the other is an ethyl group is on the order of 8.05; and the pKa of the pH-sensitive cationic lipid of the general formula (1) wherein both R³ and R⁴ are ethyl groups is on the order of 8.10. The pKa of a pH-sensitive cationic lipid wherein both R³ and R⁴ are isopropyl groups is much lower and is on the order of 6.25 (Non Patent Literature 5).

In the general formula (1), R¹ and R² are each independently a straight-chain C₁₀₋₁₄ alkyl group, a straight-chain C₁₀₋₂₀ alkenyl group having one or two unsaturated bonds, or —CH(R⁵)(R⁶), where R⁵ and R⁶ are each independently a straight-chain C₅₋₁₀ alkyl group. In the pH-sensitive cationic lipid represented by the general formula (1), when each of R¹ and R² serving as scaffolds is a relatively medium-chain alkyl group, alkenyl group, or branched alkyl group, the fluidity of the scaffolds is enhanced, introduction efficiency to NK cells is more favorable, and toxicity is also kept lower.

Examples of the straight-chain C₁₀₋₁₄ alkyl group (alkyl group having 10 to 14 carbon atoms) include a n-decyl group, a n-undecyl group, a n-dodecyl group, a n-tridecyl group, and a n-tetradecyl group. When R¹ or R² is a straight-chain C₁₀₋₁₄ alkyl group, it is preferred for the pH-sensitive cationic lipid represented by the general formula (1) that both R¹ and R² should be straight-chain C₁₀₋₁₄ alkyl groups; it is more preferred that R¹ and R² should be each independently a n-undecyl group, a n-dodecyl group, or a n-tridecyl group; it is further preferred that both R¹ and R² should be n-undecyl groups, n-dodecyl groups, or n-tridecyl groups; and it is still further preferred that both R¹ and R² should be n-tridecyl groups.

The straight-chain C₁₀₋₂₀ alkenyl group (alkenyl group having 10 to 20 carbon atoms) having one or two unsaturated bonds can be a group in which one or two saturated bonds between carbon atoms of an alkyl chain in a straight-chain C₁₀₋₂₀ alkyl group (alkyl group having 10 to 20 carbon atoms) are unsaturated bonds, and is preferably a group in which one or two saturated bonds between carbon atoms near the middle of a straight-chain C₁₀₋₂₀ alkyl group are unsaturated bonds, more preferably a group in which one or two saturated bonds between carbon atoms near the middle of a straight-chain C₁₃₋₁₈ alkyl group (alkyl group having 13 to 18 carbon atoms) are unsaturated bonds. Examples of the alkyl group having 10 to 20 carbon atoms include a n-decyl group, a n-undecyl group, a n-dodecyl group, a n-tridecyl group, a n-tetradecyl group, a n-pentadecyl group, a n-hexadecyl group, a n-heptadecyl group, a n-octadecyl group, a n-nonadecyl group, and a n-eicosyl group. The group in which one or two saturated bonds between carbon atoms near the middle of a straight-chain C₁₃₋₁₈ alkyl group are unsaturated bonds is more preferably a group in which one or two saturated bonds between carbon atoms near the middle of a n-tridecyl group, a n-tetradecyl group, a n-pentadecyl group, a n-hexadecyl group, a n-heptadecyl group, or a n-octadecyl group are unsaturated bonds. Examples thereof include a 5-tridecenyl group, a 6-tridecenyl group, a 7-tridecenyl group, a 8-tridecenyl group, a 9-tridecenyl group, a 5-tetradecenyl group, a 6-tetradecenyl group, a 7-tetradecenyl group, a 8-tetradecenyl group, a 9-tetradecenyl group, a 6-pentadecenyl group, a 7-pentadecenyl group, a 8-pentadecenyl group, a 9-pentadecenyl group, a 10-pentadecyl group, a 6-hexadecenyl group, a 7-hexadecenyl group, a 8-hexadecenyl group, a 9-hexadecenyl group, a 10-hexadecyl group, a 6-heptadecenyl group, a 7-heptadecenyl group, a 8-heptadecenyl group, a 9-heptadecenyl group, a 10-heptadecenyl group, a 11-heptadecenyl group, a 12-heptadecenyl group, a 9,12-heptadecenyl group, a 7-octadecenyl group, a 8-octadecenyl group, a 9-octadecenyl group, a 10-octadecenyl group, a 11-octadecenyl group, a 7-nonadecenyl group, a 8-nonadecenyl group, a 9-nonadecenyl group, a 10-nonadecenyl group, a 11-nonadecenyl group, a 8-eicosenyl group, a 9-eicosenyl group, a 10-eicosenyl group, a 11-eicosenyl group, and a 12-eicosenyl group.

When R¹ or R² is a straight-chain C₁₀₋₂₀ alkenyl group, it is preferred for the pH-sensitive cationic lipid represented by the general formula (1) that both R¹ and R² should be straight-chain C₁₀₋₂₀ alkenyl groups; it is more preferred that R¹ and R² should be each independently a group in which one or two saturated bonds between carbon atoms near the middle of a straight-chain C₁₃₋₁₈ alkyl group are unsaturated bonds; it is further preferred that R¹ and R² should be each independently a 6-hexadecenyl group, a 7-hexadecenyl group, a 8-hexadecenyl group, a 9-hexadecenyl group, a 10-hexadecyl group, a 6-heptadecenyl group, a 7-heptadecenyl group, a 8-heptadecenyl group, a 9-heptadecenyl group, a 10-heptadecenyl group, a 11-heptadecenyl group, a 12-heptadecenyl group, a 9,12-heptadecenyl group, a 7-octadecenyl group, a 8-octadecenyl group, a 9-octadecenyl group, a 10-octadecenyl group, or a 11-octadecenyl group; it is still further preferred that R¹ and R² should be each independently a 6-heptadecenyl group, a 7-heptadecenyl group, a 8-heptadecenyl group, a 9-heptadecenyl group, a 10-heptadecenyl group, a 11-heptadecenyl group, a 12-heptadecenyl group, or a 9,12-heptadecenyl group; and it is particularly preferred that both R¹ and R² should be 8-heptadecenyl groups.

Examples of —CH(R⁵)(R⁶), where R⁵ and R⁶ are each independently a straight-chain C₅₋₁₀ alkyl group include —CH(—C₅H₁₁)(—C₇H₁₅), —CH(—C₆H₁₃)(—C₈H₁₇), —CH(—C₇H₁₅)(—C₉H₁₉). and —CH(—C₈H₁₇) (—C₁₀H₂₁). When R¹ or R² is —CH(R⁵)(R⁶), it is preferred for the pH-sensitive cationic lipid represented by the general formula (1) that both R¹ and R² should be —CH(R⁵)(R⁶); it is more preferred that R¹ and R² should be each independently —CH(—C₅H₁₁)(—C₇H₁₅), —CH(—C₆H₁₃) (—C₈H₁₇), —CH(—C₇H₁₅) (—C₉H₁₉), or —CH(—C₈H₁₇) (—C₁₀H₂₁); and it is further preferred that both R¹ and R² should be —CH(—C₆H₁₃)(—C₈H₁₇).

The pH-sensitive cationic lipid represented by the general formula (1) is preferably a compound wherein R¹ and R² are each independently any group in which one or two saturated bonds between carbon atoms near the middle of a straight-chain C₁₃₋₁₈ alkyl group are unsaturated bonds, or a compound wherein R¹ and R² are each independently a n-undecyl group, a n-dodecyl group, or a n-tridecyl group, and p is 3 to 5, more preferably a compound wherein R¹ and R² are each independently any group in which one or two saturated bonds between carbon atoms near the middle of a straight-chain C₁₃₋₁₈ alkyl group are unsaturated bonds, and p is 3 to 5, further preferably a compound wherein R¹ and R² are each independently a 6-heptadecenyl group, a 7-heptadecenyl group, a 8-heptadecenyl group, a 9-heptadecenyl group, a 10-heptadecenyl group, a 11-heptadecenyl group, a 12-heptadecenyl group, or a 9,12-heptadecenyl group, and p is 3 to 5, still further preferably a compound wherein both R¹ and R² are 8-heptadecenyl groups, and p is 3 to 5, particularly preferably a compound wherein both R¹ and R² are 8-heptadecenyl groups, both R³ and R⁴ are methyl groups, and p is 4.

The pH-sensitive cationic lipid represented by the general formula (1) is also preferably a compound wherein R¹ and R² are each independently —CH(R⁵)(R⁶), and p is 3 to 5, more preferably a compound wherein R¹ and R² are each independently —CH(—C₅H₁₁)(—C₇H₁₅), —CH(—C₆H₁₃)(—C₈H₁₇), —CH(—C₇H₁₅)(—C₉H₁₉), or —CH(—C₈H₁₇)(—C₁₀H₂₁), and p is 3 to 5, further preferably a compound wherein both R¹ and R² are —CH(—C₆H₁₃)(—C₈H₁₇), and p is 3 to 5, still further preferably a compound wherein both R¹ and R² are —CH(—C₆H₁₃)(—C₈H₁₇), and p is 4.

The pH-sensitive cationic lipid represented by the general formula (1) can be easily produced by, for example, a method specifically shown in Examples of the present specification. With reference to this production method, those skilled in the art can easily produce an arbitrary lipid encompassed in the scope of the general formula (1) by appropriately selecting a starting compound, a reagent, and reaction conditions, etc. The pH-sensitive cationic lipid of the present invention constituting the lipid nanoparticle according to the present invention may be only one type or may be two or more types. When the pH-sensitive cationic lipid of the present invention constituting the lipid nanoparticle according to the present invention is two or more types, the amount of the pH-sensitive cationic lipid of the present invention means the total amount of lipid molecules corresponding to the pH-sensitive cationic lipid of the present invention among lipid molecules constituting the lipid nanoparticle.

A larger ratio of the pH-sensitive cationic lipid of the present invention to the lipid molecules constituting the lipid nanoparticle offers higher take-up efficiency of the lipid nanoparticle into target cells. Therefore, in the lipid nanoparticle according to the present invention, the ratio of the amount of the pH-sensitive cationic lipid of the present invention to the total amount of the lipids constituting the lipid nanoparticle ([the amount (mol) of the pH-sensitive cationic lipid of the present invention]/([the total amount (mol) of the lipids constituting the lipid nanoparticle])×100%) is preferably 20% by mol or more. On the other hand, if the ratio of the pH-sensitive cationic lipid to the lipid molecules constituting the lipid nanoparticle is too large, it may be difficult to sufficiently decrease a particle size. The ratio of the amount of the pH-sensitive cationic lipid of the present invention to the total amount of the lipids constituting the lipid nanoparticle in the lipid nanoparticle according to the present invention is more preferably 25% by mol or more, further preferably 25 to 60% by mol, still further preferably 25 to 50% by mol, particularly preferably 25 to 45% by mol, because the take-up efficiency of the lipid nanoparticle into target cells is sufficient and a lipid nanoparticle having a sufficiently small particle size can be obtained.

Among the constituent lipids in the lipid nanoparticle according to the present invention, a lipid that is generally used for forming a liposome can be used as a lipid other than the pH-sensitive cationic lipid of the present invention. Examples of such a lipid include phospholipid, sterol, glycolipid, and saturated or unsaturated fatty acid. One or two or more in combination of these lipids can be used. Examples of the phospholipid can include: glycerophospholipid such as phosphatidylserine, phosphatidylinositol, phosphatidylglycerol, phosphatidylethanolamine, phosphatidylcholine, cardiolipin, plasmalogen, ceramide phosphoryl glycerol phosphate, and phosphatidic acid; and sphingophospholipid such as sphingomyelin, ceramide phosphoryl glycerol, and ceramide phosphoryl ethanolamine. Alternatively, natural product-derived phospholipid such as egg yolk lecithin or soybean lecithin may be used. Examples of the fatty acid residue in the glycerophospholipid and sphingophospholipid can include, but are not particularly limited to, saturated or unsaturated fatty acid residues having 12 to 24 carbon atoms. A saturated or unsaturated fatty acid residue having 14 to 20 carbon atoms is preferred. Specific examples thereof can include acyl groups derived from fatty acid such as lauric acid, myristic acid, palmitic acid, palmitoleic acid, stearic acid, oleic acid, linolic acid, linolenic acid, arachidic acid, arachidonic acid, behenic acid, and lignoceric acid. When such glycerolipid or sphingolipid has two or more fatty acid residues, all the fatty acid residues may be the same group or may be groups different from each other.

Examples of the sterol include: animal-derived sterol such as cholesterol, cholesterol succinate, lanosterol, dihydrolanosterol, desmosterol, and dihydrocholesterol; plant-derived sterol (phytosterol) such as stigmasterol, sitosterol, campesterol, and brassicasterol; and microbe-derived sterol such as zymosterol and ergosterol. Examples of the glycolipid include: glyceroglycolipid such as sulfoxy ribosyl glyceride, diglycosyl diglyceride, digalactosyl diglyceride, galactosyl diglyceride, and glycosyl diglyceride; and sphingoglycolipid such as galactosyl cerebroside, lactosyl cerebroside, and ganglioside. Examples of the saturated or unsaturated fatty acid include saturated or unsaturated fatty acid having 12 to 20 carbon atoms, such as palmitic acid, oleic acid, stearic acid, arachidonic acid, and myristic acid.

In addition to the pH-sensitive cationic lipid of the present invention, a neutral lipid is preferably contained as a constituent lipid in the lipid nanoparticle according to the present invention; phospholipid or sterol is more preferably contained; sterol is further preferably contained; and cholesterol is still more preferably contained.

The lipid nanoparticle according to the present invention preferably contains a polyalkylene glycol-modified lipid as a lipid component. Polyalkylene glycol is a hydrophilic polymer, and the lipid nanoparticle is constructed using the polyalkylene glycol-modified lipid as a lipid membrane constituent lipid so that the surface of the lipid nanoparticle can be modified with the polyalkylene glycol. The surface modification with the polyalkylene glycol may be able to enhance the stability, such as retention in blood, of the lipid nanoparticle.

For example, polyethylene glycol, polypropylene glycol, polytetramethylene glycol, or polyhexamethylene glycol can be used as the polyalkylene glycol. The molecular weight of the polyalkylene glycol is, for example, on the order of 300 to 10,000, preferably on the order of 500 to 10,000, more preferably on the order of 1,000 to 5,000.

For example, stearylated polyethylene glycol (e.g., PEG45 stearate (STR-PEG45)) can be used in the modification of a lipid with polyethylene glycol. In addition, for example, a polyethylene glycol derivative such as N-[carbonyl-methoxy polyethylene glycol-2000]-1,2-dipalmitoyl-sn-glycero-3-phosphoethanolamine, n-[carbonyl-methoxy polyethylene glycol-5000]-1,2-dipalmitoyl-sn-glycero-3-phosphoethanolamine, N-[carbonyl-methoxy polyethylene glycol-750]-1,2-distearoyl-sn-glycero-3-phosphoethanolamine, N-[carbonyl-methoxy polyethylene glycol-2000]-1,2-distearoyl-sn-glycero-3-phosphoethanolamine, N-[carbonyl-methoxy polyethylene glycol-5000]-1,2-distearoyl-sn-glycero-3-phosphoethanolamine, or 1,2-dimyristoyl-rac-glycero-3-methoxy polyethylene glycol-2000 (PEG2k-DMG) may be used, though the PEGylated lipid is not limited thereto.

The ratio of the amount of the polyalkylene glycol-modified lipid to the total amount of the lipids constituting the lipid nanoparticle according to the present invention is not particularly limited as long as the amount does not impair high introduction efficiency to NK cells brought about by the pH-sensitive cationic lipid of the present invention, specifically, NK cell-specific gene expression activity when the lipid nanoparticle according to the present invention is used as a gene carrier. The ratio of the amount of the polyalkylene glycol-modified lipid to the total amount of the lipids constituting the lipid nanoparticle is, for example, preferably 0.5 to 3% by mol.

The lipid nanoparticle according to the present invention can be subjected, if necessary, appropriate surface modification.

The retention in blood of the lipid nanoparticle according to the present invention can be enhanced by modifying its surface with a hydrophilic polymer or the like. Use of a lipid modified with such a modifying group as a constituent lipid in the lipid nanoparticle may be able to perform surface modification.

In the production of the lipid nanoparticle according to the present invention, for example, glycophorin, ganglioside GM1, phosphatidylinositol, ganglioside GM3, a glucuronic acid derivative, a glutamic acid derivative, or a polyglycerin phospholipid derivative may be used as a lipid derivative for enhancing retention in blood. Alternatively, polyalkylene glycol as well as dextran, pullulan, Ficoll, polyvinyl alcohol, a styrene-maleic anhydride alternate copolymer, a divinyl ether-maleic anhydride alternate copolymer, amylose, amylopectin, chitosan, mannan, cyclodextrin, pectin, carrageenan, or the like may be used as a hydrophilic polymer for enhancing retention in blood in surface modification.

In order to promote the nuclear localization of the lipid nanoparticle according to the present invention, the lipid nanoparticle may be surface-modified with, for example, a trisaccharide or higher oligosaccharide compound. The type of the trisaccharide or higher oligosaccharide compound is not particularly limited. For example, an oligosaccharide compound in which approximately 3 to 10 sugar units are bonded can be used, and preferably, an oligosaccharide compound in which approximately 3 to 6 sugar units are bonded can be used. Among others, preferably, an oligosaccharide compound which is a trimer to a hexamer of glucose can be used, and more preferably, an oligosaccharide compound which is a trimer or a tetramer of glucose can be used. More specifically, for example, isomaltotriose, isopanose, maltotriose, maltotetraose, maltopentaose, or maltohexaose can be suitably used. Among them, maltotriose, maltotetraose, maltopentaose, or maltohexaose in which glucose units are linked through an α1-4 bond is more preferred. Maltotriose or maltotetraose is particularly preferred, and maltotriose is most preferred. The amount of the oligosaccharide compound modifying the surface of the lipid nanoparticle is not particularly limited and is, for example, on the order of 1 to 30% by mol, preferably on the order of 2 to 20% by mol, more preferably on the order of 5 to 10% by mol, based on the total amount of the lipids.

The method for surface-modifying the lipid nanoparticle with the oligosaccharide compound is not particularly limited. For example, a liposome in which the surface of a lipid nanoparticle is modified with a monosaccharide such as galactose or mannose (International Publication No. WO 2007/102481) is known. Therefore, a surface modification method described in this publication can be adopted. The disclosure of the publication is incorporated herein by reference in its entirety.

For example, any one or two or more functions such as a temperature change-sensitive function, a membrane permeation function, a gene expression function, and a pH-sensitive function can be imparted to the lipid nanoparticle according to the present invention. These functions appropriately imparted thereto improve the retention of the lipid nanoparticle in blood and allow the lipid nanoparticle to efficiently escape from endosome after endocytosis in target cells so that an encapsulated nucleic acid can be intracellularly expressed in NK cells with higher efficiency.

The lipid nanoparticle according to the present invention may contain one or two or more substances selected from the group consisting of an antioxidant such as tocopherol, propyl gallate, ascorbyl palmitate, or butylated hydroxytoluene, a charged substance, and a membrane polypeptide. Examples of the charged substance that confers positive charge can include saturated or unsaturated aliphatic amine such as stearylamine and oleylamine. Examples of the charged substance that confers negative charge can include dicetyl phosphate, cholesteryl hemisuccinate, phosphatidylserine, phosphatidylinositol, and phosphatidic acid. Examples of the membrane polypeptide include peripheral membrane polypeptides and integral membrane polypeptides. The amount of such a substance blended is not particularly limited and can be appropriately selected according to a purpose.

The size of the lipid nanoparticle according to the present invention is preferably an average particle size of 400 nm or smaller, more preferably an average particle size of 300 nm or smaller, further preferably an average particle size of 200 nm or smaller, still further preferably 150 nm or smaller, because high delivery efficiency to in vivo NK cell can be easily obtained. The average particle size of the lipid nanoparticle means a number-average particle size measured by dynamic light scattering (DLS). The measurement by dynamic light scattering can be performed by a routine method using a commercially available DLS apparatus or the like.

The polydispersity index (PDI) of the lipid nanoparticle according to the present invention is on the order of 0.01 to 0.7, preferably on the order of 0.01 to 0.5, more preferably on the order of 0.03 to 0.2. A zeta potential can be set to the range of 1 mV to 20 mV, preferably the range of 5 mV to 15 mV.

Examples of the form of the lipid nanoparticle according to the present invention can include, but are not particularly limited to, forms dispersed in an aqueous solvent, such as unilamellar liposomes, multilamellar liposomes, spheric micelles, and amorphous layered structures. The lipid nanoparticle according to the present invention is preferably a unilamellar liposome or a multilamellar liposome.

It is preferred for the lipid nanoparticle according to the present invention that a component of interest to be delivered into target cells should be included inside the particle surrounded by a lipid membrane. The component that is included inside the lipid nanoparticle according to the present invention is not particularly limited as long as its size allows the component to be included. An arbitrary substance such as a nucleic acid, a saccharide, a peptide, a low-molecular compound, or a metal compound can be encapsulated in the lipid nanoparticle according to the present invention.

The component that is included in the lipid nanoparticle according to the present invention is preferably a nucleic acid. The nucleic acid may be DNA, may be RNA, or may be an analog or a derivative thereof (e.g., peptide nucleic acid (PNA) or phosphorothioate DNA). The nucleic acid that is included in the lipid nanoparticle according to the present invention may be a single-stranded nucleic acid, may be double-stranded nucleic acid, may be linear, or may be circular.

The nucleic acid that is included in the lipid nanoparticle according to the present invention preferably contains a foreign gene to be expressed in target cells, and is more preferably a nucleic acid that functions so as to express a foreign gene in cells when taken up into the cells. The foreign gene may be a gene that is originally contained in the genomic DNA of target cells (preferably NK cells) or may be a gene that is not contained in the genomic DNA. Examples of such a nucleic acid include gene expression vectors containing a nucleic acid having a nucleotide sequence encoding a gene of interest to be expressed. The gene expression vector may reside as an extrachromosomal gene in the recipient cells or may be taken up into genomic DNA by homologous recombination.

The gene expression vector that is included in the lipid nanoparticle according to the present invention is not particularly limited. For example, a vector that is generally used in gene therapy can be used. The gene expression vector that is included in the lipid nanoparticle according to the present invention is preferably a nucleic acid vector such as a plasmid vector. The plasmid vector may be circular or may be cut into a linear form in advance and encapsulated in this state in the lipid nanoparticle according to the present invention. The gene expression vector can be designed by a routine method using a molecular biological tool generally used on the basis of nucleotide sequence information on a gene to be expressed, and can be produced by various methods known in the art.

The nucleic acid that is included in the lipid nanoparticle according to the present invention is also preferably a functional nucleic acid that controls the expression of a target gene present in target cells. Examples of the functional nucleic acid include antisense oligonucleotides, antisense DNA, antisense RNA, siRNA, microRNA, and mRNA. Alternatively, plasmid DNA (pDNA) serving as an siRNA expression vector for intracellular expression of siRNA may be used. The siRNA expression vector may be prepared from a commercially available siRNA expression vector, or this siRNA expression vector may be appropriately engineered. The nucleic acid that is included in the lipid nanoparticle according to the present invention is preferably mRNA or pDNA, particularly, because introduction efficiency to NK cells is favorable.

The method for producing the lipid nanoparticle according to the present invention is not particularly limited, and an arbitrary method available to those skilled in the art can be adopted. As one example, the lipid nanoparticle can be produced by dissolving all lipid components in an organic solvent such as chloroform, performing drying under reduced pressure using an evaporator or spray drying using a spray dryer to form a lipid membrane, then adding an aqueous solvent containing a component, for example, a nucleic acid, to be encapsulated in the lipid nanoparticle to the dried mixture thus obtained, and further emulsifying the mixture using an emulsifier such as a homogenizer, an ultrasonic emulsifier, or a high-pressure jet emulsifier. Alternatively, the lipid nanoparticle may be produced by a method well known as a method for producing a liposome, for example, a reverse-phase evaporation method. In the case of controlling the size of the lipid nanoparticle, extrusion (extrusion filtration) can be performed at a high pressure using a membrane filter having a pore size, for example.

Examples of the composition of the aqueous solvent (dispersion medium) can include, but are not particularly limited to, buffer solutions such as phosphate buffer solutions, citrate buffer solutions, and phosphate-buffered saline, saline, and media for cell cultures. Such an aqueous solvent (dispersion medium) can stably disperse the lipid nanoparticle and may be further supplemented with, for example, a sugar (aqueous solution) including monosaccharides such as glucose, galactose, mannose, fructose, inositol, ribose, and xylose sugar, disaccharides such as lactose, sucrose, cellobiose, trehalose, and maltose, trisaccharides such as raffinose and melezitose, polysaccharides such as cyclodextrin, and sugar alcohols such as erythritol, xylitol, sorbitol, mannitol, and maltitol, or a polyhydric alcohol (aqueous solution) such as glycerin, diglycerin, polyglycerin, propylene glycol, polypropylene glycol, ethylene glycol, diethylene glycol, triethylene glycol, polyethylene glycol, ethylene glycol monoalkyl ether, diethylene glycol monoalkyl ether, or 1,3-butylene glycol. For stable long-term preservation of the lipid nanoparticle dispersed in this aqueous solvent, it is desirable to eliminate an electrolyte as much as possible from the aqueous solvent, in terms of physical stability such as suppression of aggregation. It is also desirable to set the pH of the aqueous solvent to weakly acidic to nearly neutral pH (pH on the order of 3.0 to 8.0) and/or to remove dissolved oxygen by nitrogen bubbling or the like, in terms of chemical stability of the lipids.

The lipid nanoparticle according to the present invention may be produced by an alcoholic dilution method using a channel. This method is a method of producing the lipid nanoparticle by introducing a solution of lipid components dissolved in an alcoholic solvent and a solution of water-soluble components (which are to be included in the lipid nanoparticle) dissolved in an aqueous solvent from separate channels, and combining these solutions. A lipid nanoparticle having a diameter on the order of 30 nm can be reproducibly produced by using a microchannel with a built-in three-dimensional micromixer capable of achieving instantaneous mixing of two liquids (Non Patent Literature 6). The channel for use in production is preferably a channel structure having a simple two-dimensional structure, as described in Patent Literature 2, having a microsized channel where a starting material solution flows, and baffles (baffle plates) with a given width with respect to a channel width which are arranged in a staggered manner on both sides, because a nanosized lipid particle formation system having high particle size controllability can be formed. The aqueous solvent described above can be used in the alcoholic dilution method.

In the case of freeze-drying or spray-drying the obtained aqueous dispersion of the lipid nanoparticle, use of, for example, a sugar (aqueous solution) including monosaccharides such as glucose, galactose, mannose, fructose, inositol, ribose, and xylose sugar, disaccharides such as lactose, sucrose, cellobiose, trehalose, and maltose, trisaccharides such as raffinose and melezitose, polysaccharides such as cyclodextrin, and sugar alcohols such as erythritol, xylitol, sorbitol, mannitol, and maltitol, may be able to improve stability. In the case of freezing the aqueous dispersion, use of, for example, the sugar described above or a polyhydric alcohol (aqueous solution) such as glycerin, diglycerin, polyglycerin, propylene glycol, polypropylene glycol, ethylene glycol, diethylene glycol, triethylene glycol, polyethylene glycol, ethylene glycol monoalkyl ether, diethylene glycol monoalkyl ether, or 1,3-butylene glycol may be able to improve stability.

Owing to this gene expression activity highly selective for NK cells, the lipid nanoparticle according to the present invention functions as a gene expression carrier targeting NK cells. The lipid nanoparticle according to the present invention has favorable delivery efficiency to NK cells and as such, is suitably used in, for example, a method for transforming NK cells. In the transformation of NK cells, a lipid nanoparticle containing a foreign gene of interest to be expressed in an NK cell, or a functional nucleic acid that controls gene expression in an NK cell is used as the lipid nanoparticle according to the present invention. In the description below, the gene to be expressed in an NK cell and the functional nucleic acid that controls gene expression in an NK cell are also collectively referred to as a “NK cell-targeting nucleic acid”. When the lipid nanoparticle according to the present invention containing the NK cell-targeting nucleic acid is introduced to NK cells, the NK cells are transformed with the NK cell-targeting nucleic acid contained in the lipid nanoparticle. When the NK cell-targeting nucleic acid contained in the introduced lipid nanoparticle is the gene to be expressed in an NK cell, this gene is expressed in the transformed NK cells. When the NK cell-targeting nucleic acid contained in the introduced lipid nanoparticle is the functional nucleic acid that controls gene expression in an NK cell, the expression of a target gene of the functional nucleic acid is suppressed in the transformed NK cells.

The lipid nanoparticle according to the present invention and an NK cell for use in transforming the NK cell may be combined to produce a kit for transforming the NK cell. Owing to this kit, the NK cell transformation can be more conveniently carried out.

The foreign gene of interest to be expressed in an NK cell or the functional nucleic acid that controls gene expression in an NK cell is encapsulated in the lipid nanoparticle according to the present invention, which is then administered to a test animal so that the foreign gene or the functional nucleic acid contained in the lipid nanoparticle can be expressed in NK cells of the test animal. When the lipid nanoparticle according to the present invention in which a gene expression vector is encapsulated is administered to an animal individual, the gene expression vector encapsulated in the lipid nanoparticle is expressed in NK cells with high efficiency. For example, when the lipid nanoparticle according to the present invention in which the foreign gene of interest to be expressed in an NK cell is encapsulated is administered to a test animal, the foreign gene can be expressed in NK cells of the test animal. Likewise, when the lipid nanoparticle according to the present invention in which the functional nucleic acid or an siRNA expression vector is encapsulated is administered to an animal individual, the functional nucleic acid or the siRNA expression vector encapsulated in the lipid nanoparticle is expressed in NK cells in the body of the animal with high efficiency so that the expression of a gene targeted by the nucleic acid is suppressed.

The lipid nanoparticle according to the present invention can be used as an active ingredient in a pharmaceutical composition. The lipid nanoparticle according to the present invention is excellent as a gene carrier and as such, is useful as an active ingredient in a pharmaceutical composition for use in gene therapy, particularly, gene therapy targeting NK cells. The lipid nanoparticle is also useful as an active ingredient in a pharmaceutical composition for use in cancer treatment.

NK cells are cells that play an important role in immune functions. Therefore, the lipid nanoparticle according to the present invention serving as a gene carrier highly selective for NK cells can be used as an active ingredient in a pharmaceutical composition for use in immunotherapy and is suitable, particularly, as an active ingredient in a pharmaceutical composition for use in cancer immunotherapy. Examples of the cancer immunotherapy include NK cell therapy and cancer vaccine therapy.

An NK cell transfected with the lipid nanoparticle according to the present invention (transformed NK cell) is also useful as an active ingredient in a pharmaceutical composition. This transformed NK cell may be used as an NK cell that is administered to a patient in NK cell therapy. For example, the lipid nanoparticle according to the present invention containing a NK cell-targeting nucleic acid that contributes to NK cell activation is introduced to NK cells ex vivo, and the obtained transformed NK cells are administered to an animal having a cancer tissue. The transformed NK cells are activated by the expression of the introduced NK cell-targeting nucleic acid, and the transformed NK cells thus activated attack cancer tissues in the body of the animal. As a result, the cancer tissues in the body of the animal can be reduced in size, or increase in size thereof can be suppressed, thereby suppressing the cancer.

The animal to which the lipid nanoparticle according to the present invention or the transformed NK cell transfected with the same is administered is not particularly limited and may be a human or may be an animal other than a human. Examples of the nonhuman animal include mammals such as bovines, pigs, horses, sheep, goats, monkeys, dogs, cats, rabbits, mice, rats, hamsters, and guinea pigs, and birds such as chickens, quails, and ducks. The administration route in administering the lipid nanoparticle according to the present invention to the animal is not particularly limited and is preferably parenteral administration such as transvenous administration, enteral administration, intramuscular administration, subcutaneous administration, transcutaneous administration, transnasal administration, or transpulmonary administration.

### EXAMPLES

Next, the present invention will be described in more detail with reference to Examples. However, the present invention is not limited by the following Examples.

<Lipid>

In experiments given below, the following lipids were used as pH-sensitive cationic lipids.

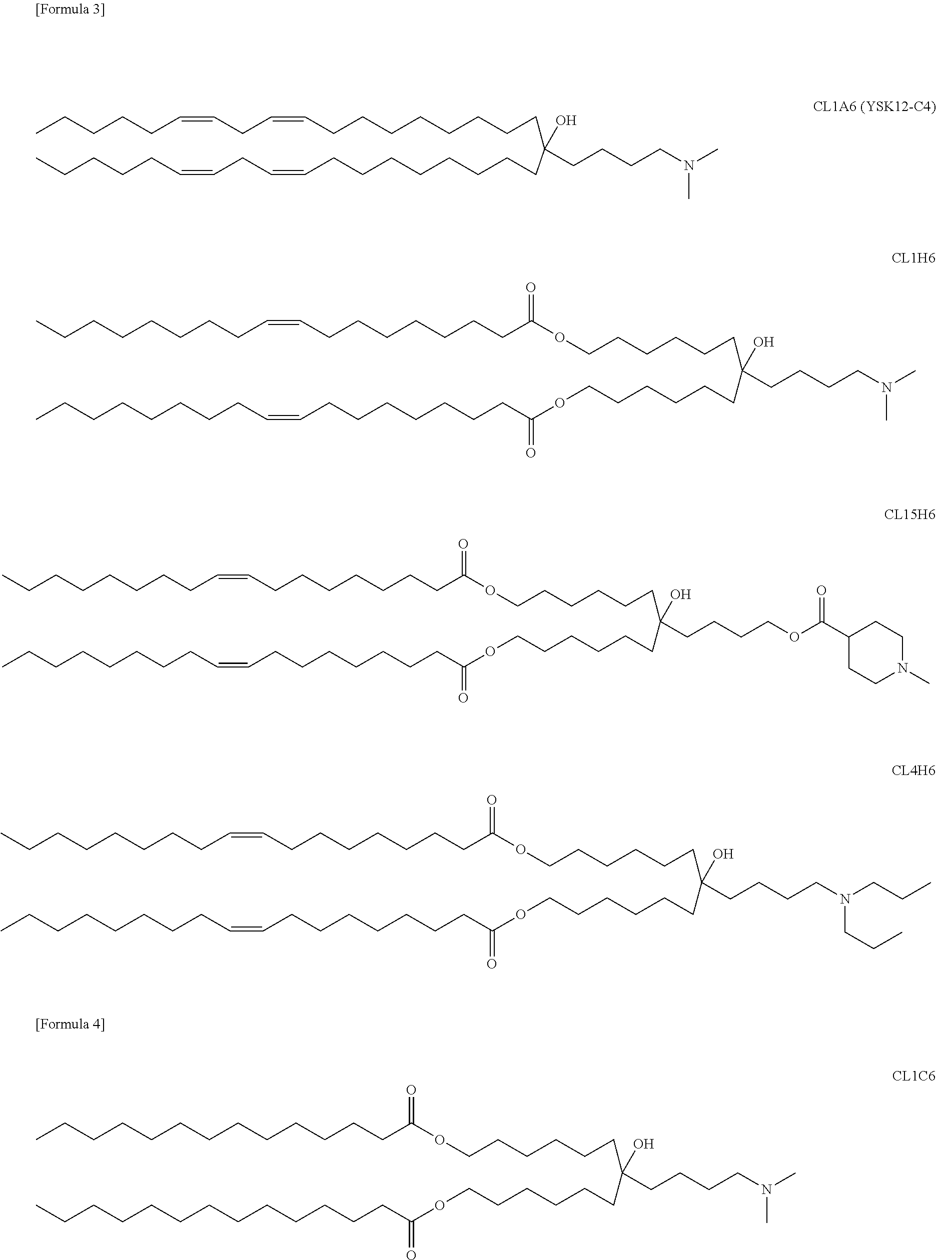

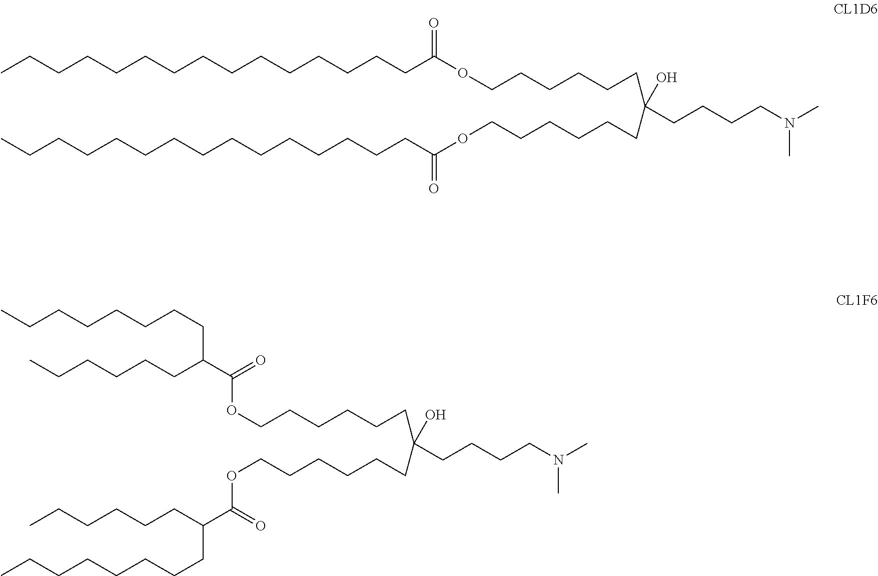

CL1A6 (YSK12-C4), CL1H6, CL15H6, CL4H6, CL1C6, and CL1D6 used were synthesized by the method described in Patent Literature 1.

As for CL1F6, 7-(4-(dimethylamino)butyl)tridecane-1,7,13-triol (1.30 mmol) synthesized by the method described in Patent Literature 1 was dissolved in 5 mL of dichloromethane. Subsequently, 2-hexyldecanoic acid (3.12 mmol), DMAP (N,N-dimethyl-4-aminopyridine) (0.26 mmol) and EDCI (1-ethyl-3-(3-dimethylaminopropyl)carbodiimide) (3.9 mmol) were added thereto, and the mixture was reacted overnight at room temperature. The solvent was distilled off using a rotary evaporator. Then, the residue was suspended in ethyl acetate, and insoluble matter was filtered off. The filtrate was subjected to extraction with a 0.5 N aqueous sodium oxide solution and saturated saline. The organic layer was dehydrated by the addition of anhydrous sodium sulfate. After filtration of the resultant, the solvent was distilled off using a rotary evaporator to obtain a crude product. The crude product was subjected to silica gel chromatography [eluting solvent; dichloromethane:methanol (continuous gradient)], and the purified product thus obtained was used.

Further, cholesterol (chol) and polyethylene glycol 2000-modified dimyristoyl glycerol (PEG2k-DMG) were used as neutral lipids.

<Preparation of Lipid Nanoparticle>

In experiments given below, lipid nanoparticles were prepared by use of the t-butanol dilution method unless otherwise specified.

Specifically, a t-butanol solution containing all lipid components was first prepared as a lipid solution. Subsequently, an siRNA or mRNA solution was added in small portions to the prepared lipid solution with stirring using a vortex mixer. A 1 mL syringe equipped with a 27 G injection needle was packed with the mixed solution, which was then injected from the syringe to 2 mL of a 20 mM citrate buffer solution (pH 6.0). Then, the citrate buffer solution with the lipid solution injected from the syringe was diluted by the addition of D-PBS(−) (pH 8.0) while ultrafiltration (1000×g, 20° C., 8 min) was performed using Amicon Ultra-15 centrifugal filter device (MWCO 100,000). Subsequently, 12 mL of D-PBS(−) (pH 8.0) was added thereto, followed by ultrafiltration (1000×g, 20° C., 8 min) again. Then, the liposome solution was recovered into a 1.5 mL tube while thoroughly washed with D-PBS(−). The solution was diluted into 1 mL with D-PBS(−), and the resultant was used as a lipid nanoparticle solution.

<Preparation of Lipofectamine-mRNA Complex>

Lipofectamine Messenger Max Reagent and an mRNA solution were each diluted into a predetermined concentration with a culture medium (Opti-MEM), mixed with each other at a ratio of 1:1 using a vortex mixer, and left standing at room temperature for 5 minutes for preparation.

<Measurement of Average Particle Size and Zeta Potential of Lipid Nanoparticle>

The average particle size (average number) of lipid nanoparticles in PBS(−) and the zeta potential thereof in a 10 mM HEPES buffer solution (pH 7.4) were measured using an analysis apparatus “Zetasizer Nano ZS ZEN3600” (manufactured by Malvern Panalytical Ltd.) which exploits dynamic light scattering.

<Measurement of pKa of Lipid Nanoparticle>

The pKa of lipid nanoparticles was measured using p-toluenesulfonic acid (TNS). TNS (final concentration: 0.75 mM) and the lipid nanoparticles (final concentration: 30 mM) were first mixed in a buffer solution adjusted to each pH. The fluorescence intensity of the prepared mixed solution was measured using a microplate reader. The highest and lowest values among the measurement values were regarded as 100% and 0% charge rates, respectively, and pH that exhibited a 50% charge rate was calculated as pKa.

<Nucleic Acid Encapsulation Rate of Lipid Nanoparticle>

The nucleic acid (siRNA or mRNA) encapsulation rate of lipid nanoparticles was quantified by assay using “Quant-iT(R) RiboGreen(R) RNA” (manufactured by Thermo Fisher Scientific Inc.) which emits fluorescence by causing intercalation selectively for RNA.

The prepared lipid nanoparticle solution was mixed with a 10 mM HEPES buffer, 10% triton (if no triton was added, an equal amount of a 10 mM HEPES buffer was added), and RiboGreen, added to each well of a 96-well plate, and shaken for 5 minutes in a mixer for a shaker. Then, the plate was placed in a microplate reader (“EnSpire”, manufactured by PerkinElmer, Inc.), and the fluorescence intensity of the solution in each well was measured.

The nucleic acid encapsulation rate was calculated according to the following expression.

[Encapsulation rate (%)]=([Nucleic acid concentration in triton(+)]−[Nucleic acid concentration in triton(−)])/[Nucleic acid concentration in triton(+)]×100

<Cell Culture>

A human NK cell line NK-92 was passaged by using change in color of a medium from red to orange colors as a guideline. The cells were recovered into a 50 mL tube. A supernatant was removed by centrifugation (130×g, 4° C., 5 min). After counting of the cells, the cells were added at a concentration of 2×10⁶ cells/10 mL to a flask, and human recombinant IL-2 was added thereto at a final concentration of 200 U/mL, followed by culture at 37° C. under a condition of 5% CO2.

A human NK cell line KHYG-1 was passaged by using change in color of a medium from red to orange colors as a guideline. The passage of the cells was performed by mere dilution. Specifically, the cells were diluted 3 to 4-fold. Then, human recombinant IL-2 was added thereto at a final concentration of 200 U/mL, followed by culture at 37° C. under a condition of 5% CO2.

A human NK cell line NK-92MI was cultured in the same manner as in the NK-92 cells except that no human recombinant IL-2 was added to a medium.

A human malignant melanoma cell line A375 was passaged by using change in color of a medium from red to orange colors as a guideline. The cells were recovered into a 50 mL tube. A supernatant was removed by centrifugation (130×g, 4° C., 5 min). The cells were inoculated at a passage ratio of 1:3 to 1:5 to a 10 cm dish and cultured at 37° C. under a condition of 5% CO2.

<Evaluation of knockdown activity of siGAPDH- or siSMAD3-encapsulated lipid nanoparticle>

A NK cell line (NK-92 or NK-92MI) was transfected with each lipid nanoparticle. The knockdown activity of the siGAPDH- or siSMAD3-encapsulated lipid nanoparticle was examined by RT-qPCR using RNA extracted from the cells 24 hours later as a template.

First, the NK cell line was recovered into a 50 mL tube and centrifuged (130×g, 4° C., 5 min). The supernatant was removed, and the cells were suspended in 10 mL of a culture medium (Opti-MEM), counted, and then resuspended into 4×10⁶ cells/mL in a culture medium. To this cell suspension, the siGAPDH- or siSMAD3-encapsulated lipid nanoparticle was added at an siRNA concentration of 10 nM, 30 nM, 60 nM, or 90 nM, and further, human recombinant IL-2 was added at a final concentration of 200 U/mL. Then, the mixture was dispensed at 4×10⁵ or 8×10⁵ cells/well to a plate for suspension culture (MS-8012R). The plate was incubated at 37° C. for 2 hours under a condition of 5% CO2. Then, a serum-containing medium (IL-2: 200 U/mL) was added at 500 μL/well, and the plate was incubated at 37° C. for 22 hours under a condition of 5% C02. In the case of the NK-92MI cells, no IL-2 was added to the medium.

The cells were recovered into a 1.5 mL tube from each well and centrifuged (800×g, 4° C., 3 min). After removal of the supernatant, 1 mL of D-PBS(−) was added to the precipitated cells, which were then centrifuged (800×g, 4° C., 3 min). After removal of the supernatant, 350 μL of an RLT buffer (1% 2-mercaptoethanol) was added to the cells, which were then stirred using a vortex mixer and then transferred to a 2 mL safe-lock tube. Subsequently, RNA was purified using a kit for RNA purification (“RNeasy Mini Kit”, manufactured by Qiagen N.V.). Then, absorbance was measured using micro-volume spectrophotometer “Nano drop” (manufactured by Thermo Fisher Scientific Inc.) to measure the concentration and purity of the RNA.

Reverse-transcription reaction was performed with the recovered RNA as a template using an oligo dT primer and a random primer (6 mars) in a kit for reverse-transcription reaction (“PrimeScript RT reagent kit”, manufactured by Takara Bio Inc.). Then, a GAPDH or SMAD3 mRNA level was measured by real-time PCR with the obtained cDNA as a template using relative quantification based on the ΔΔCt method and β-actin (ACTB) as an endogenous gene. The ratio of the GAPDH or SMAD3 mRNA level to the ACTB mRNA level ([GAPDH or SMAD3 mRNA level]/[ACTB mRNA level]) was regarded as the knockdown activity of the siGAPDH- or siSMAD3-encapsulated lipid nanoparticle.

<Evaluation of Toxicity of siGAPDH- or siSMAD3-Encapsulated Lipid Nanoparticle>

The toxicity of the siGAPDH- or siSMAD3-encapsulated lipid nanoparticle was examined by WST-1 assay using a NK cell line (NK-92 or NK-92MI) transfected with each lipid nanoparticle.

First, the NK cell line was recovered into a 50 mL tube and centrifuged (130×g, 4° C., 5 min). The supernatant was removed, and the cells were suspended in 10 mL of a culture medium (Opti-MEM), counted, and then resuspended into 4×10⁶ cells/mL in a culture medium. To this cell suspension, the siGAPDH- or siSMAD3-encapsulated lipid nanoparticle was added at an siRNA concentration of 10 nM, 30 nM, 60 nM, or 90 nM. Then, the mixture was dispensed at 1.2×10⁵ or 2.4×10⁵ cells/well to two wells of a 96-well plate. The plate was incubated at 37° C. for 2 hours under a condition of 5% CO2. Then, a serum-containing medium (IL-2: 200 U/mL) was added at 50 μL/well, and the plate was incubated at 37° C. for 22 hours under a condition of 5% CO₂. In the case of the NK-92MI cells, no IL-2 was added to the medium.

Subsequently, 10 or 20 μL of a premix for reaction (“Premix WST-1 Cell Proliferation Assay System”, manufactured by Takara Bio Inc.) was added to each well, and the plate was incubated at 37° C. for 0.5 to 1 hours under a condition of 5% CO₂. Then, absorbance was measured using a plate reader (“Varioskan LUX”, manufactured by Thermo Fisher Scientific Inc.). The survival rate (%) of the cells was calculated according to the following expression.

[Survival rate (%)]=([Absorbance value of the cells supplemented with the siGAPDH- or siSMAD3−encapsulated lipid nanoparticle]−[Base absorbance value])/([Absorbance value of the cells non-supplemented with the siGAPDH- or siSMAD3-encapsulated lipid nanoparticle]−[Base absorbance value])×100(%)

<Evaluation of Toxicity of mRNA-Encapsulated Lipid Nanoparticle>

NK-92 cells or NK-92MI cells were recovered into a 50 mL tube and centrifuged (130×g, 4° C., 5 min). The supernatant was removed, and the cells were suspended in 10 mL of a culture medium (Opti-MEM), counted, and then resuspended into 4×10⁶ cells/mL in a culture medium (Opti-MEM). Each mRNA-encapsulated lipid nanoparticle or a Lipofectamine-mRNA complex was prepared at an mRNA concentration of 0.066 μg/mL, 0.2 μg/mL, 0.4 μg/mL, or 0.6 μg/mL using a 1.5 mL tube and inoculated to a 96-well plate (1.2×10⁵ cells/well, n=2), and the plate was incubated at 37° C. for 2 hours under a condition of 5% CO₂. Then, a serum-containing medium (IL-2: 200 U/mL) was added at 50 μL/well, and the plate was incubated at 37° C. for 22 hours under a condition of 5% CO₂. In the case of the NK-92MI cells, no IL-2 was added to the medium.

Subsequently, a premix for reaction (“Premix WST-1 Cell Proliferation Assay System”, manufactured by Takara Bio Inc.) was added thereto (10 μL/well), and the plate was incubated at 37° C. for 1 hour under a condition of 5% CO₂. Absorbance was measured using a plate reader (“Varioskan LUX”). The survival rate (%) of the cells was calculated according to the following expression.

[Survival rate (%)]=([Absorbance value of the cells supplemented with the mRNA-encapsulated lipid nanoparticle]−[Base absorbance value])/([Absorbance value of the cells non-supplemented with the mRNA-encapsulated lipid nanoparticle]−[Base absorbance value])×100(%)

<Evaluation of Ability to Deliver Luc mRNA>

NK-92 cells or NK-92MI cells were recovered into a 50 mL tube and centrifuged (130×g, 4° C., 5 min). The supernatant was removed, and the cells were suspended in 10 mL of a culture medium (Opti-MEM), counted, and then resuspended into 4×10⁶ cells/mL in a culture medium. To this cell suspension, each Luc mRNA (trade name: “CleanCap FLuc mRNA”, manufactured by TriLink BioTechnologies)-encapsulated lipid nanoparticle or a Lipofectamine-mRNA complex was added at an mRNA concentration of 0.066 μg/mL, 0.2 μg/mL, or 0.4 μg/mL. The mixture was dispensed (4×10⁵ cells/well, IL-2: 200 U/mL) to a plate for suspension culture (MS-8012R), and the plate was incubated at 37° C. for 2 hours under a condition of 5% C02. Then, a serum-containing medium (IL-2: 200 U/mL) was added at 500 μL/well, and the plate was incubated at 37° C. for 22 hours under a condition of 5% C02. In the case of the NK-92MI cells, no IL-2 was added to the medium.

The cells were recovered into a 1.5 mL tube from each well and centrifuged (800×g, 4° C., 3 min). After removal of the supernatant, 1 mL of D-PBS(−) was added to the cells, which were then centrifuged (800 g, 4° C., 3 min). After removal of the supernatant, 100 μL of Passive Lysis Buffer was added to each tube, which were then stirred using a vortex mixer for 30 seconds and then centrifuged (17800×g, 4° C., 2 min). 70 μL of the supernatant was recovered into a separately provided 1.5 mL tube.

The amount of luminescence of luciferase was evaluated by luciferase assay using a luciferase assay system. 50 μL of a luciferase assay substrate dissolved in a luciferase assay buffer was added to a Rohren tube, and 20 μL of the supernatant obtained by the centrifugation mentioned above was added thereto. After pipetting, the amount of luminescence was measured in a luminometer. The obtained supernatant was also used in BCA assay using Pierce BCA PROTEIN Assay Kit. The protein concentration of each sample was calculated, and the amount of luminescence was corrected with the protein level.

<Evaluation of Ability to Deliver GFP mRNA>

NK-92 cells or NK-92MI cells were recovered into a 50 mL tube and centrifuged (130×g, 4° C., 5 min). The supernatant was removed, and the cells were suspended in 10 mL of a culture medium (Opti-MEM), counted, and then resuspended into 4×10⁶ cells/mL in a culture medium (Opti-MEM). To this cell suspension, each GFP mRNA (trade name: “CleanCap EGFP mRNA”, manufactured by TriLink BioTechnologies)-encapsulated lipid nanoparticle or a Lipofectamine-mRNA complex was added at an mRNA concentration of 0.066 μg/mL, 0.2 μg/mL, or 0.4 μg/mL. The mixture was dispensed (4×10⁵ cells/well, IL-2: 200 U/mL) to a plate for suspension culture (MS-8012R), and the plate was incubated at 37° C. for 2 hours under a condition of 5% C02. Then, a serum-containing medium (IL-2: 200 U/mL) was added at 500 μL/well, and the plate was incubated at 37° C. for 22 hours under a condition of 5% C02. In the case of the NK-92MI cells, no IL-2 was added to the medium.

The cells were recovered into a 1.5 mL tube from each well and centrifuged (800×g, 4° C., 3 min). After removal of the supernatant, 1 mL of D-PBS(−) was added to the cells, which were then centrifuged (800×g, 4° C., 3 min). After removal of the supernatant, 1 mL of a FACS buffer was added to the cells, which were then centrifuged (800×g, 4° C., 3 min) to remove a supernatant. Then, the cells were suspended in 500 to 1000 μL of a FACS buffer, and the expression of GFP was analyzed using Cyto FLEX.

<GFP Observation Under Fluorescence Microscope>

NK-92MI was recovered into a 50 mL tube and centrifuged (130×g, 4° C., 5 min). The supernatant was removed, and the cells were suspended in 10 mL of a culture medium (Opti-MEM), counted, and then resuspended into 4×10⁶ cells/mL in a culture medium (Opti-MEM). To this cell suspension, each EGFP mRNA (trade name: “CleanCap EGFP mRNA”, manufactured by TriLink BioTechnologies)-encapsulated lipid nanoparticle was added at an mRNA concentration of 0.2 μg/mL. The mixture was dispensed (4×10⁵ cells/well) to a plate for suspension culture (MS-8012R), and the plate was incubated at 37° C. for 2 hours under a condition of 5% CO₂. Then, a serum-containing medium was added at 500 μL/well, and the plate was incubated at 37° C. for 22 hours under a condition of 5% CO₂.

The cells were recovered into a 1.5 mL tube from each well and centrifuged (800×g, 4° C., 3 min). After removal of the supernatant, 1 mL of D-PBS(−) was added to the cells, which were then centrifuged (800×g, 4° C., 3 min). After removal of the supernatant, Prolong Diamond was added (10 μL/tube), and the mixture was transferred to a glass slide and observed under a microscope (BZ-X800).

### Example 1

In order to examine the influence of apparent pKa of a pH-sensitive cationic lipid constituting a lipid nanoparticle on siRNA introduction efficiency and cytotoxicity to NK-92 cells, siRNA-encapsulated lipid nanoparticles were prepared using YSK12-C4 (pKa: 8.2), CL1H6 (pKa: 8.2), CL15H6 (pKa: 7.3), and CL4H6 (pKa: 6.35) and introduced to NK-92 cells.

A lipid nanoparticle was prepared by encapsulating siRNA against GAPDH (siGAPDH) in a lipid nanoparticle having a content ratio of the pH-sensitive cationic lipid ([the amount (mol) of the pH-sensitive cationic lipid]/([the total amount (mol) of the lipids constituting the lipid nanoparticle])×100, %) of 50% by mol. Specifically, a t-butanol solution containing the pH-sensitive cationic lipid, cholesterol, and PEG2k-DMG with composition having a molar ratio of 50:50:1 and having a total lipid concentration of 1.25 mM was used as a lipid solution, and a 3 μM aqueous siGAPDH solution was used as RN. An siGAPDH-encapsulated lipid nanoparticle was prepared by the method of the section <Preparation of lipid nanoparticle>.

The siGAPDH-encapsulated lipid nanoparticle having composition (YSK12/cholesterol/PEG2k-DMG=85/15/1 (molar ratio)) described in Non Patent Literature 3 was similarly prepared as a subject to be compared.

The physical properties of the prepared lipid nanoparticle having a pH-sensitive cationic lipid content ratio of 50% by mol were measured (n=2). The results are shown in Table 1.

TABLE 1

| | Particle | | Zeta | siRNA | siRNA |
|---|---|---|---|---|---|
| | size | | potential | encapsulation | recovery rate |
| Lipid | (nm) | PDI | (mV) | rate (%) | (%) |
| CL1A6 | 143 | 0.051 | 9.9 | 97.6 | 103.4 |
| (YSK12-C4) | | | | | |
| CL1H6 | 141 | 0.095 | 10.1 | 102.3 | 102.8 |
| CL15H6 | 134 | 0.068 | 6.38 | 95.1 | 108.8 |
| CL4H6 | 116 | 0.075 | 4.69 | 94.5 | 99.3 |

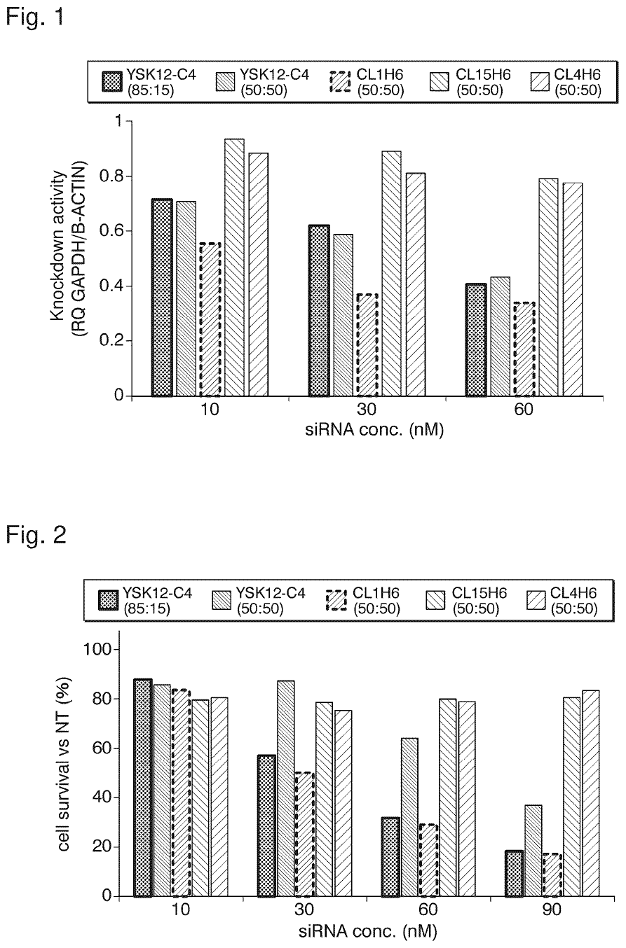

Each siGAPDH-encapsulated lipid nanoparticle was introduced to NK-92 cells, and knockdown activity ([GAPDH mRNA level]/[ACTB mRNA level]) was measured (n=1). The measurement results are shown in FIG. 1. In the drawing, “YSK12-C4 (85:15)” depicts the results about the siGAPDH-encapsulated lipid nanoparticle having a YSK12-C4 content ratio of 85% by mol (YSK12-LNP (85%)); “YSK12-C4 (50:50)” depicts the results about the siGAPDH-encapsulated lipid nanoparticle having a YSK12-C4 content ratio of 50% by mol (YSK12-LNP (50%)); “CL1H6 (50:50)” depicts the results about the siGAPDH-encapsulated lipid nanoparticle having a CL1H6 content ratio of 50% by mol (CL1H6-LNP (50%)); “CL15H6 (50:50)” depicts the results about the siGAPDH-encapsulated lipid nanoparticle having a CL15H6 content ratio of 50% by mol (CL15H6-LNP (50%)); and “CL4H6 (50:50)” depicts the results about the siGAPDH-encapsulated lipid nanoparticle having a CL4H6 content ratio of 50% by mol (CL4H6-LNP (50%)).

As shown in FIG. 1, CL1H6-LNP (50%) exhibited higher gene knockdown activity than that of YSK12-LNP (85%). By contrast, CL15H6-LNP (50%) and CL4H6-LNP (50%) exhibited no activity. These results suggested that use of CL1H6 as a constituent lipid in the lipid nanoparticle can enhance gene knockdown activity against NK cell lines.

Subsequently, NK-92 cells were transfected with each siGAPDH-encapsulated lipid nanoparticle, and cytotoxicity 24 hours later was examined by WST-1 assay (n=1). FIG. 2 shows results of measuring the survival rate (%) of the cells transfected with each siGAPDH-encapsulated lipid nanoparticle. As a result, CL1H6-LNP (50%) exhibited cytotoxicity equivalent to that of YSK12-LNP (85%). By contrast, CL15H6-LNP (50%) and CL4H6-LNP (50%) hardly exhibited toxicity. CL15H6-LNP (50%) and CL4H6-LNP (50%) exhibited neither knockdown activity nor toxicity, suggesting that these lipid nanoparticles were not taken up by NK cell lines in the first place.

### Example 2

An siGAPDH-encapsulated lipid nanoparticle was prepared by changing the ratio between CL1H6 and cholesterol as to CL1H6-LNP (50%) prepared in Example 1, and influence on gene knockdown activity and cytotoxicity was tested.

An siGAPDH-encapsulated lipid nanoparticle was prepared in the same manner as in Example 1 except that each t-butanol solution containing CL1H6, cholesterol, and PEG2k-DMG with composition having a molar ratio of 15:85:1, 25:75:1, 35:65:1, or 50:50:1 and having a total lipid concentration of 1.25 mM was used as a lipid solution.

Likewise, an siGAPDH-encapsulated lipid nanoparticle was prepared in the same manner as in Example 1 except that each t-butanol solution containing YSK12-C4, cholesterol, and PEG2k-DMG with composition having a molar ratio of 15:85:1, 25:75:1, 35:65:1, or 50:50:1 and having a total lipid concentration of 1.25 mM was used as a lipid solution.

The physical properties of the siGAPDH-encapsulated lipid nanoparticle are shown in Table 2 (n=3) and Table 3 (n=2 or 3).

TABLE 2

| Lipid composition | | | | siRNA | |
|---|---|---|---|---|---|
| (mol %) | Particle size | | Zeta potential | encapsulation | siRNA recovery |
| (CL1H6/Chol) | (nm) | PDI | (mV) | rate (%) | rate (%) |
| 15/85 | 151 ± 5 | 0.39 ± 0.068 | 9.8 ± 0.7 | 95.6 ± 0.7 | 79.2 ± 7.4 |
| 25/75 | 127 ± 3 | 0.13 ± 0.008 | 11.6 ± 0.4 | 96.7 ± 0.9 | 105.4 ± 3.6 |
| 35/65 | 126 ± 3 | 0.15 ± 0.015 | 11.3 ± 1.2 | 96.4 ± 0.9 | 106.5 ± 2.1 |
| 50/50 | 152 ± 2 | 0.05 ± 0.011 | 13.5 ± 1.3 | 97.7 ± 0.6 | 112.0 ± 1.0 |

TABLE 3

| Lipid | | | | | |
|---|---|---|---|---|---|
| com- | | | | | |
| position | | | | SIRNA | SIRNA |
| (mol %) | Particle | | Zeta | encapsu- | recovery |
| (YSK12- | size | | potential | lation | rate |
| C4/Chol) | (nm) | PDI | (mV) | rate (%) | (%) |
| 15/85 | 203 | 0.49 | 5.5 | 96.6 | 55.2 |
| 25/75 | 167 ± 1.4 | 0.24 ± 0.03 | 7.6 ± 0.6 | 96.7 ± 1.0 | 91.7 ± 2.1 |
| 35/65 | 145 | 0.17 | 8.4 | 97.1 | 98.4 |
| 50/50 | 154 | 0.09 | 8.6 | 96.7 | 108.6 |

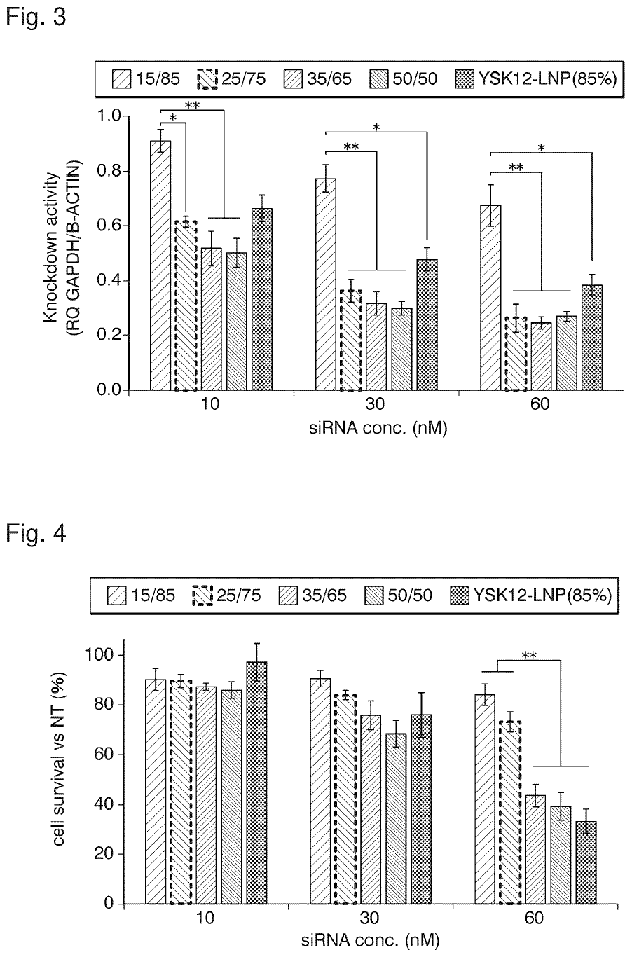

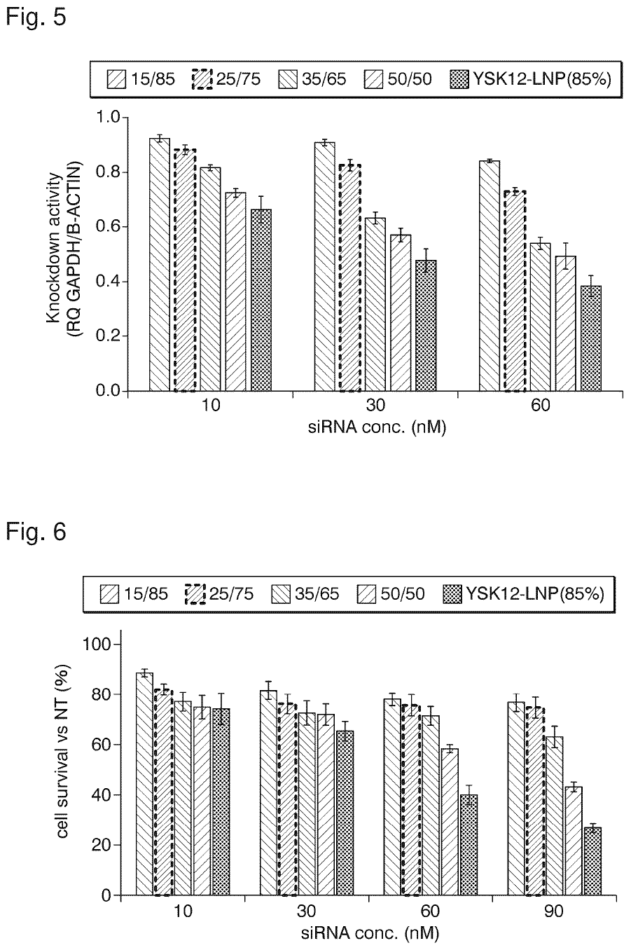

Knockdown activity and cytotoxicity were measured as to each siGAPDH-encapsulated lipid nanoparticle in the same manner as in Example 1. These items were also similarly measured as to YSK12-LNP (85%) prepared in Example 1 as a subject to be compared. The results of knockdown activity of CL1H6-LNP are shown in FIG. 3, the results of cytotoxicity of CL1H6-LNP are shown in FIG. 4, the results of knockdown activity of YSK12-LNP are shown in FIG. 5, and the results of cytotoxicity of YSK12-LNP are shown in FIG. 6. In FIG. 3 (n=3), “**” represents P<0.01, and “*” represents P<0.05. In FIG. 4 (n=4), “**” represents P<0.01 (based on ANOVA followed by the Tukey-Kramer method).

As shown in FIG. 3, CL1H6-LNP containing 25% by mol or more of CH1H6 exhibited gene knockdown activity equivalent to or higher than that of YSK12-LNP (85%). As shown in FIG. 4, CL1H6-LNP having 25% by mol or less of CL1H6 was found to have little cytotoxicity. These results demonstrated that use of a lipid nanoparticle having a CL1H6/cholesterol molar ratio of 25/75 as a gene carrier can achieve siRNA introduction with high knockdown activity and low toxicity.

As shown in FIGS. 5 and 6, in the case of YSK12-LNP, gene knockdown activity was reduced with decrease in ratio of YSK12-C4, and cytotoxicity was also reduced in response thereto. The phenomenon, observed in CL1H6-LNP, in which toxicity was able to be reduced, without impairing activity, with decrease in content ratio of CL1H6 was not observed in YSK12-LNP.

The results about CL1H6-LNP (25%) containing 25% by mol of CH1H6 were analyzed and compared as CL1H6-LNP excellent both in gene knockdown activity and in cytotoxicity with the results about YSK12-LNP (85%) containing 85% by mol of YSK12-C4 and the results about YSK12-LNP (25%) containing 25% by mol of YSK12-C4.

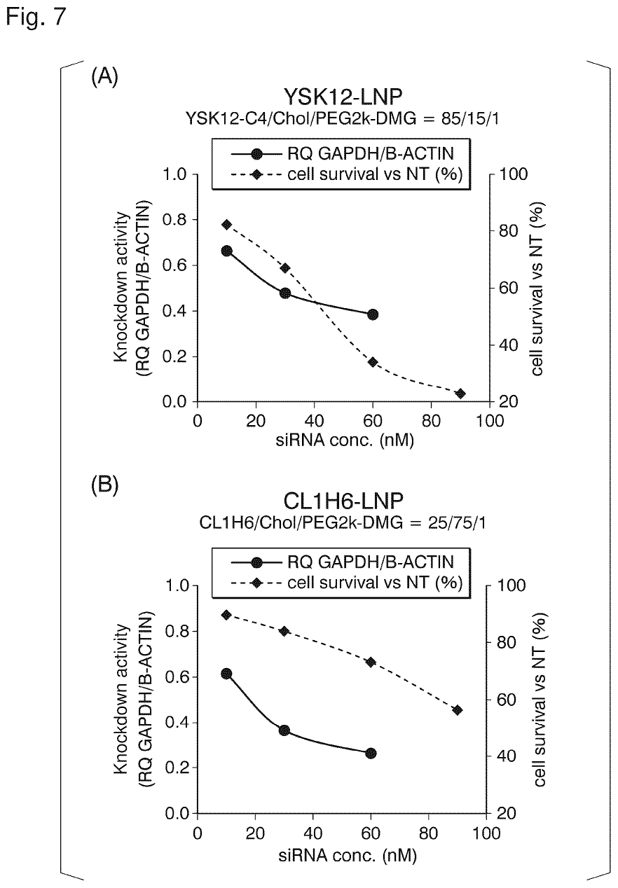

The gene knockdown activity and cell survival rate of YSK12-LNP (85%) and CL1H6-LNP (25%) were plotted with an siRNA concentration at the time of transfection on the abscissa and gene knockdown activity and a cell survival rate (%) on the ordinate. FIG. 7(A) shows the results about YSK12-LNP (85%), and FIG. 7(B) shows the results about CL1H6-LNP (25%). As shown in FIG. 7, CL1H6-LNP (25%) was found to succeed in the dissociation between gene knockdown activity and cytotoxicity.

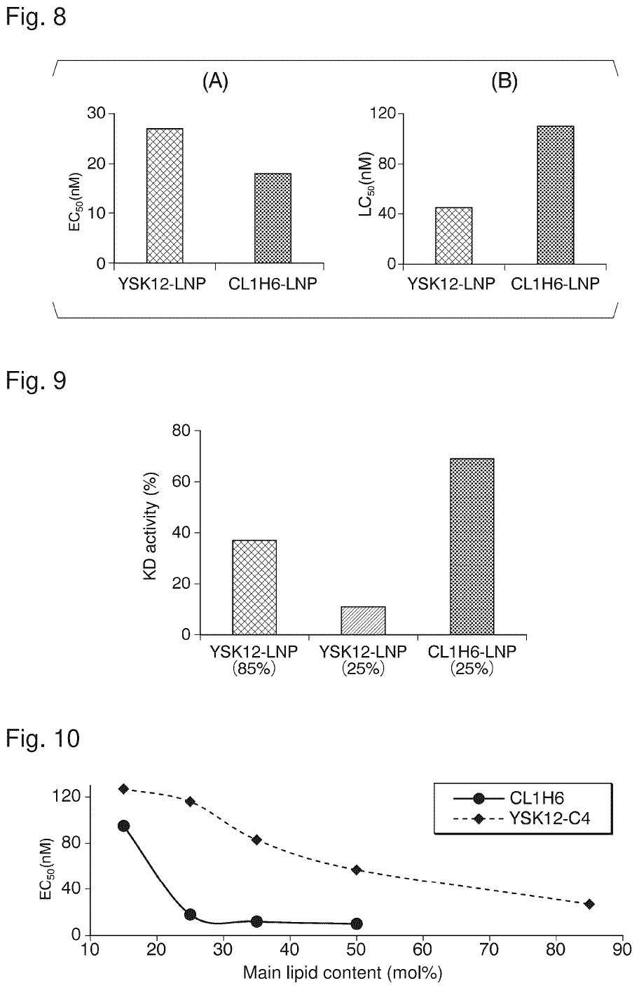

A half maximal effective concentration (EC₅₀) (nM) (FIG. 8(A)) and a median lethal concentration (LC₅₀) (nM) (FIG. 8(B)) were compared between YSK12-LNP (25%) and CL1H6-LNP (25%). As a result, CL1H6-LNP (25%) decreased EC₅₀ to approximately 0.67 times and increased LC₅₀ even to approximately 2.5 times as compared with YSK12-LNP (25%). When gene knockdown activity at a cell survival rate of 80% was compared therebetween, CL1H6-LNP (25%) exhibited the highest gene knockdown activity of 70% or more (FIG. 9). As a result of plotting with a YSK12-C4 or CL1H6 content ratio (% by mol) on the abscissa and EC₅₀ (nM) on the ordinate, CL1H6 was found to be able to exhibit high gene knockdown activity at a small content ratio (FIG. 10).

### Example 3

As shown in Example 1, the pH-sensitive cationic lipid contained in the lipid nanoparticle that exhibited high gene knockdown activity against NK-92 cells was YSK12-C4 and CL1H6 which had the same hydrophilic moiety. These results suggested that pKa around 8.2 of the lipid nanoparticle is suitable for achieving high gene knockdown activity against NK cells. Accordingly, the influence of the structure of a scaffold moiety of CL1H6 on gene knockdown activity was examined.

An siGAPDH-encapsulated lipid nanoparticle having a CL1C6 content ratio of 25% by mol (CL1C6-LNP (25%)) and an siGAPDH-encapsulated lipid nanoparticle having a CL1D6 content ratio of 25% by mol (CL1D6-LNP (25%)) were prepared in the same manner as in Example 2 except that: CL1C6 or CL1D6 was used instead of CL1H6; the 20 mM citrate buffer solution (pH 6.0) for injecting the mixed solution of the lipid solution and the siRNA solution from syringe was replaced with a 5 mM citrate buffer solution (pH 6.0, 60° C.); and D-PBS(−) for use in dilution was replaced with D-PBS(−) (pH 8.5, 60° C.). CL1C6-LNP (25%) and YSK-LNP (85%) used were prepared in the same manner as in Example 2.

The physical properties of each siGAPDH-encapsulated lipid nanoparticle are shown in Table 4 (n=3).

TABLE 4

| | | | | SIRNA | siRNA |
|---|---|---|---|---|---|
| | Particle | | Zeta | encapsu- | recovery |
| | size | | potential | lation | rate |
| Lipid | (nm) | PDI | (mV) | rate (%) | (%) |
| CL1A6 | 152 ± 8 | 0.055 ± 0.006 | 10.5 ± 0.3 | 96.7 ± 0.4 | 114.9 ± 1.4 |
| (YSK12- | | | | | |
| C4) | | | | | |
| CL1H6 | 127 ± 3 | 0.130 ± 0.008 | 11.6 ± 0.4 | 96.7 ± 0.9 | 105.4 ± 3.6 |
| CL1C6 | 138 ± 9 | 0.120 ± 0.013 | 11.0 ± 0.9 | 85.8 ± 0.5 | 120.9 ± 7.4 |
| CL1D6 | 142 ± 7 | 0.120 ± 0.003 | 11.3 ± 0.4 | 83.0 ± 1.2 | 109.2 ± 2.5 |

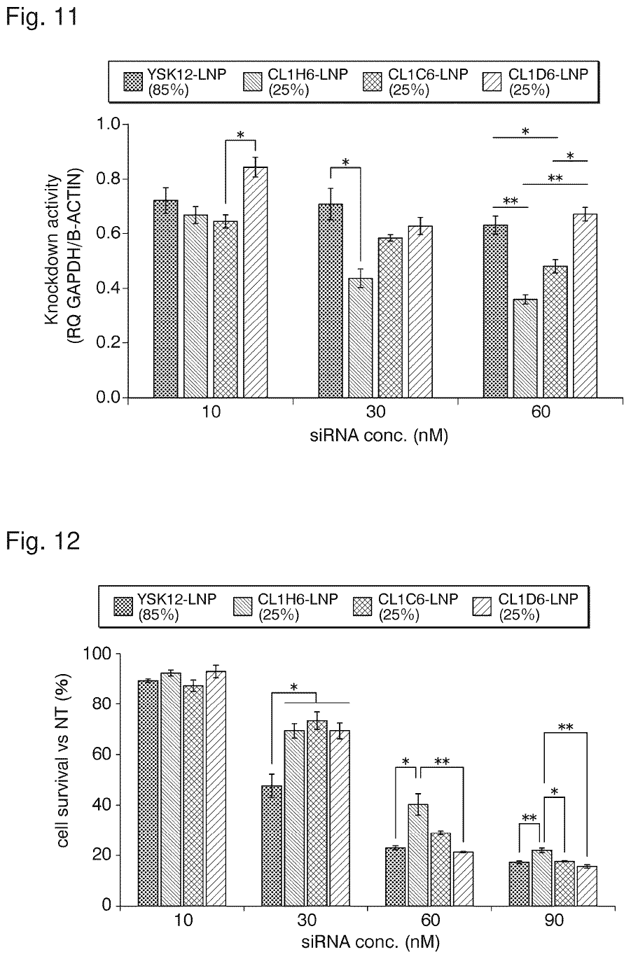

Knockdown activity and cytotoxicity were measured as to each siGAPDH-encapsulated lipid nanoparticle in the same manner as in Example 1 using NK-92 cells. The results of knockdown activity of each siGAPDH-encapsulated lipid nanoparticle are shown in FIG. 11, and the results of cytotoxicity thereof are shown in FIG. 12. In both the drawings (n=3), “**” represents P<0.01, and “*” represents P<0.05 (based on ANOVA followed by the Tukey-Kramer method).

As shown in FIG. 11, CL1H6-LNP (25%) exhibited the highest gene knockdown activity. CL1C6-LNP (25%) had higher gene knockdown activity than that of CL1D6-LNP (25%), demonstrating that when a scaffold is a saturated hydrocarbon chain, the length of the chain influences gene knockdown activity. As shown in FIG. 12, CL1H6-LNP (25%) had the highest toxicity-alleviating effect.

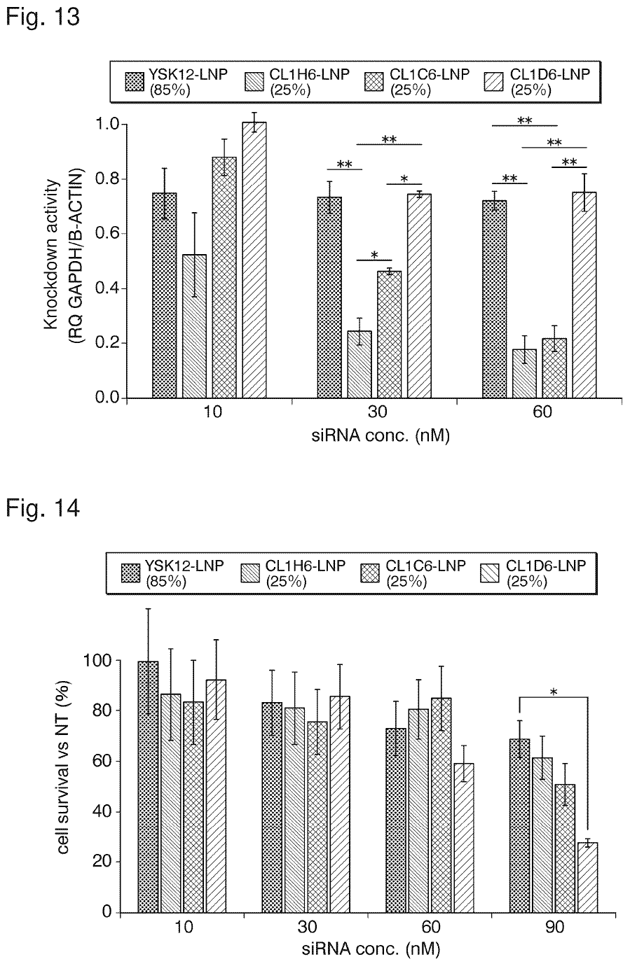

In order to test efficacy on a NK cell line other than NK-92 cells, knockdown activity and cytotoxicity were measured as to each siGAPDH-encapsulated lipid nanoparticle in the same manner as above using another human NK cell line KHYG-1 cells. The results of knockdown activity of each siGAPDH-encapsulated lipid nanoparticle are shown in FIG. 13, and the results of cytotoxicity thereof are shown in FIG. 14. In both the drawings (n=3), “**” represents P<0.01, and “*” represents P<0.05 (based on ANOVA followed by the Tukey-Kramer method).

As shown in FIG. 13, CL1H6-LNP (25%) also exhibited the highest gene knockdown activity against KHYG-1 cells. On the other hand, in cytotoxicity evaluation, no marked toxicity was observed in CL1H6-LNP (25%) (FIG. 14). These results suggested the possibility that CL1H6-LNP is useful for various human NK cell lines.

### Example 4

A lipid nanoparticle in which siGAPDH or siSMAD3 was encapsulated in CL1H6 (pKa: 8.2) was introduced to NK-92MI cells and examined for its possibility of gene knockdown in the NK-92MI cells and cytotoxicity to the NK-92MI cells.

(Preparation of CL1H6-LNP)

The following solutions were mixed to prepare an siRNA solution.

| | |
|---|---|
| 50 μM siSMAD3 solution or siGAPDH solution | 12 μL |
| RNase free water | 88 μL |

Solutions of the following components dissolved in t-BuOH were mixed to prepare a lipid solution. The lipid composition was CL1H6/cholesterol/PEG2k-DMG=25/75/1.

| | |
|---|---|
| CL1H6 (10 mM) | 12.5 μL |
| Cholesterol (2 mM) | 187.5 μL |
| PEG2k-DMG (0.2 mM) | 25 μL |
| t-BuOH (90%) | 175 μL |

An siGAPDH- or siSMAD3-encapsulated lipid nanoparticle was prepared by the method of the section <Preparation of lipid nanoparticle>using the prepared siRNA solution and lipid solution. The physical properties of the lipid nanoparticle (n=3, mean±SE) are shown in Table 5.

TABLE 5

| | | | | Encapsu- | |
|---|---|---|---|---|---|
| SIRNA- | Particle | | Zeta | lation | Recovery |
| encapsulated | size | | potential | rate | rate |
| CL1H6-LNP | (nm) | PDI | (mV) | (%) | (%) |
| SiGAPDH | 127 ± 3 | 0.130 ± 0.008 | 11.6 ± 0.4 | 96.7 ± 0.9 | 105.4 ± 3.6 |
| siSMAD3 | 126 ± 6 | 0.158 ± 0.030 | 8.3 ± 1.1 | 83.7 ± 0.3 | 94.3 ± 0.6 |

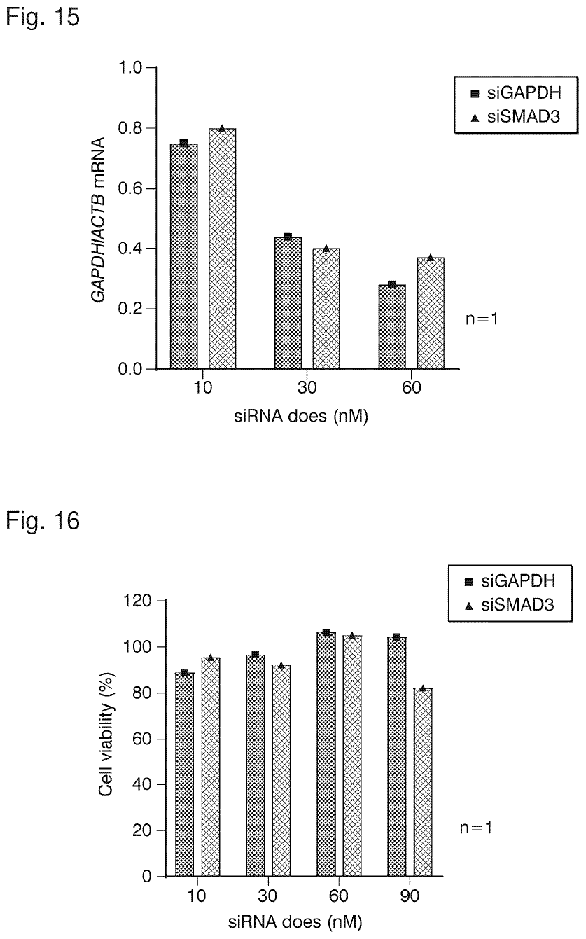

Each siRNA-encapsulated lipid nanoparticle was introduced to NK-92MI cells, and knockdown activity ([GAPDH or SMAD3 mRNA level]/[ACTB mRNA level]) was measured (n=1). The measurement results are shown in FIG. 15. In the drawing, “siGAPDH” depicts the results about the siGAPDH-encapsulated lipid nanoparticle (CL1H6-LNP), and “siSMAD3” depicts the results about the siSMAD3-encapsulated lipid nanoparticle (CL1H6-LNP).

As shown in FIG. 15, each siRNA-encapsulated CL1H6-LNP also exhibited high gene knockdown activity against NK-92MI cells. The siGAPDH-encapsulated CL1H6-LNP and the siSMAD3-encapsulated CL1H6 exhibited the same level of knockdown activity.

Subsequently, NK-92MI cells were transfected with each siRNA-encapsulated lipid nanoparticle, and cytotoxicity 24 hours later was examined by WST-1 assay (n=1). FIG. 16 shows results of measuring the survival rate (%) of the cells transfected with each siRNA-encapsulated lipid nanoparticle. As shown in FIG. 16, the siGAPDH-encapsulated CL1H6-LNP and the siSMAD3-encapsulated CL1H6 exhibited no cytotoxicity at the concentrations found to exert gene knockdown activity.

### Example 5

BALB/cSlc-nu/nu mice in which A375 cells were subcutaneously transplanted to the flank (5×10⁶ cells/70 μL/mouse, 26 G needle) were provided. PBS, untreated NK-92MI cells, and NK-92MI cells transfected on the previous day with siSMAD3-encapsulated CL1H6-LNP (siSMAD3 concentration: 30 nM) were respectively administered to the tail veins of the mice (5×10⁶ cells/200 μL/mouse, 26 G needle). The administration was performed twice a week a total of 6 times. Specifically, the administration was performed on days 7, 10, 14, 17, 21, and 24 from tumor transplantation. The major axis and minor axis of tumor were measured over time, and a tumor volume was calculated according to the following mathematical expression.

Tumor volume(mm³)=Major axis×Minor axis×Minor axis×0.52

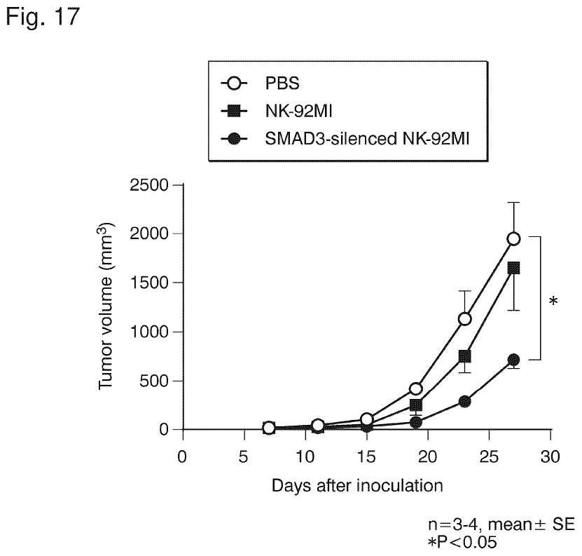

The major axis and minor axis of tumor were measured on days 7, 11, 15, 19, 23, and 27. The results are shown in FIG. 17. As shown in FIG. 17, significant antitumor activity was found in the group given NK-92MI cells in which SMAD3 was knocked down using CL1H6-LNP.

### Example 6

Luc mRNA-encapsulated lipid nanoparticles were prepared using YSK12-C4, CL1H6, CL1C6, CL1D6 and Dlin-MC3.

### Preparation of YSK12-LNP

The following solutions were mixed to prepare a Luc mRNA solution.

| | |
|---|---|
| Luc mRNA solution (1 mg/mL) | 3.95 μL |
| RNase free water | 186.05 μL |

Solutions of the following components dissolved in t-BuOH were mixed to prepare a lipid solution. The lipid composition was YSK12/cholesterol/PEG2k-DMG=85/15/1.

| | |
|---|---|
| YSK12-C4 (10 mM) | 42.5 μL |
| Cholesterol (2 mM) | 37.5 μL |
| PEG2k-DMG (0.2 mM) | 25 μL |
| t-BuOH (90%) | 295 μL |

A luciferase-encapsulated lipid nanoparticle was prepared by the method of the section <Preparation of lipid nanoparticle>using the prepared Luc mRNA solution and lipid solution. The physical properties of the lipid nanoparticle (n=3, mean±SE) are shown in Table 6.

### Preparation of CL1H6-LNP

The following solutions were mixed to prepare a Luc mRNA solution.

| | |
|---|---|
| Luc mRNA solution (1 mg/mL) | 3.95 μL |
| RNase free water | 86.05 μL |

Solutions of the following components dissolved in t-BuOH were mixed to prepare a lipid solution. The lipid composition was CLH6/cholesterol/PEG2k-DMG=25/75/1.

| | |
|---|---|
| CL1H6 (10 mM) | 12.5 μL |
| Cholesterol (2 mM) | 187.5 μL |
| PEG2k-DMG (0.2 mM) | 25 μL |
| t-BuOH (90%) | 175 μL |

A luciferase-encapsulated lipid nanoparticle was prepared by the method of the section <Preparation of lipid nanoparticle>using the prepared Luc mRNA solution and lipid solution. The physical properties of the lipid nanoparticle (n=3, mean±SE) are shown in Table 6.

(Preparation of CL1C6-LNP and CL1D6-LNP)

The following solutions were mixed to prepare a Luc mRNA solution.

| | |
|---|---|
| Luc mRNA solution (1 mg/mL) | 3.95 μL |
| RNase free water | 86.05 μL |

Solutions of the following components dissolved in t-BuOH were mixed to prepare a lipid solution. The lipid composition was (CL1C6 or CL1D6)/cholesterol/PEG2k-DMG=25/75/1.

| | |
|---|---|
| CL1C6 or CL1D6 (10 mM) | 12.5 μL |
| Cholesterol (2 mM) | 187.5 μL |
| PEG2k-DMG (0.2 mM) | 25 μL |
| t-BuOH (90%) | 175 μL |

A luciferase-encapsulated lipid nanoparticle was prepared by the method of the section <Preparation of lipid nanoparticle>using the prepared Luc mRNA solution and lipid solution. The physical properties of the lipid nanoparticle (n=3, mean±SE) are shown in Table 6.

(Preparation of MC3-LNP (N/P=8))

The following solutions were mixed to prepare a Luc mRNA solution.

| | |
|---|---|
| Luc mRNA solution (1 mg/mL) | 10 μL |
| RNase free water | 90 μL |

Solutions of the following components dissolved in t-BuOH were mixed to prepare a lipid solution. The lipid composition was Dlin-MC3/DSPC/cholesterol/PEG2k-DMG=25/75/1.

| | |
|---|---|
| Dlin-MC3 (10 mM) | 25 μL |
| Cholesterol (2 mM) | 97.5 μL |
| DSPC (2 mM) | 25 μL |
| PEG2k-DMG (0.5 mM) | 15 μL |
| t-BuOH (90%) | 212.5 μL |

A luciferase-encapsulated lipid nanoparticle was prepared by the method of the section <Preparation of lipid nanoparticle>using the prepared Luc mRNA solution and lipid solution. The physical properties of the lipid nanoparticle (n=3, mean±SE) are shown in Table 6.

TABLE 6

| Luc mRNA- | | | | Encapsulation | Recovery |
|---|---|---|---|---|---|
| encapsulated | Particle | | Zeta potential | rate | rate |
| LNP | size (nm) | PDI | (mV) | (%) | (%) |
| YSK12-LNP | 145 ± 16 | 0.141 ± 0.047 | 8.1 ± 1.8 | 73.0 ± 3.4 | 117.5 ± 6.0 |
| CL1H6-LNP | 132 ± 11 | 0.301 ± 0.081 | 11.9 ± 1.7 | 70.8 ± 5.9 | 125.7 ± 16.2 |
| CL1C6-LNP | 182 ± 7 | 0.256 ± 0.007 | 13.6 ± 0.5 | 75.3 ± 3.1 | 121.4 ± 1.9 |
| CL1D6-LNP | 172 ± 7 | 0.234 ± 0.042 | 9.5 ± 2.4 | 63.0 ± 2.4 | 86.7 ± 9.2 |
| MC3-LNP | 116 ± 0.5 | 0.074 ± 0.004 | −5.2 ± 1.3 | 74.7 ± 0.8 | 113.9 ± 7.3 |

(Preparation of Lipofectamine-mRNA Complex)

A Lipofectamine-mRNA complex (in the drawing, “LF mMAX”) was prepared by the method described in the section <Preparation of Lipofectamine-mRNA complex>using Luc mRNA as mRNA.

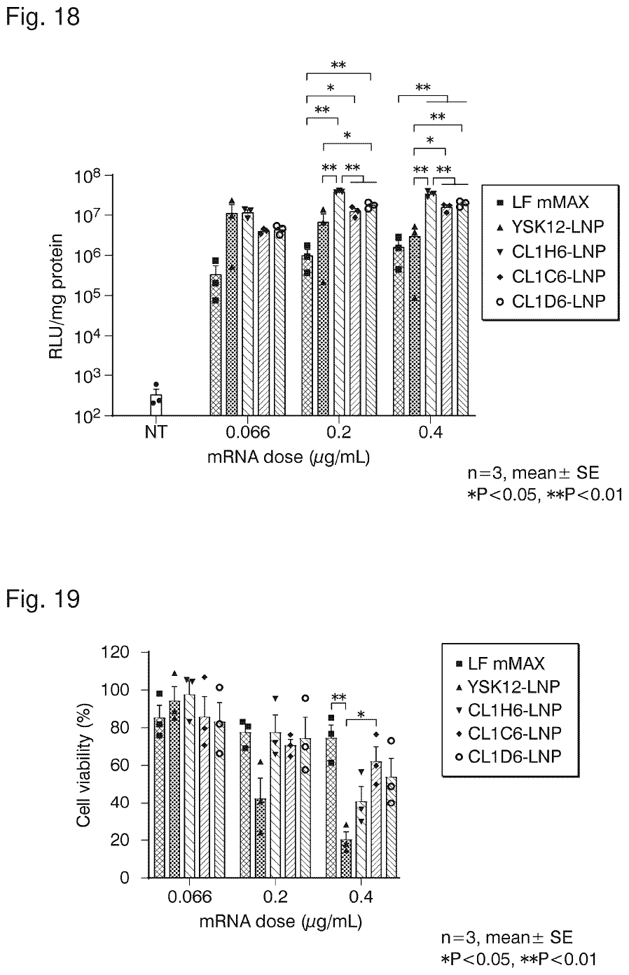

NK-92 cells were transfected with each Luc mRNA-encapsulated lipid nanoparticle or the Lipofectamine-mRNA complex, and luciferase activity 24 hours later was examined by the method described in the section <Evaluation of ability to deliver Luc mRNA>. The measurement results are shown in FIG. 18.

As shown in FIG. 18, all the Luc mRNA-encapsulated lipid nanoparticles exhibited higher luciferase activity than that of the Lipofectamine-mRNA complex. Among them, CL1H6-LNP exhibited the highest luciferase activity.

Subsequently, NK-92 cells were transfected with each Luc mRNA-encapsulated lipid nanoparticle or the Lipofectamine-mRNA complex, and cytotoxicity 24 hours later was examined by the method described in the section <Evaluation of toxicity of mRNA-encapsulated lipid nanoparticle>. The measurement results are shown in FIG. 19.

As shown in FIG. 19, CL1H6-LNP, CL1C6-LNP and CL1D6-LNP exhibited a high cell survival rate and no cytotoxicity at the concentrations that sufficiently produced luciferase activity.

### Example 7

Luc mRNA-encapsulated MC3-LNP, Luc mRNA-encapsulated CL1H6-LNP, and a Lipofectamine-mRNA complex were prepared by the same method as in Example 6.

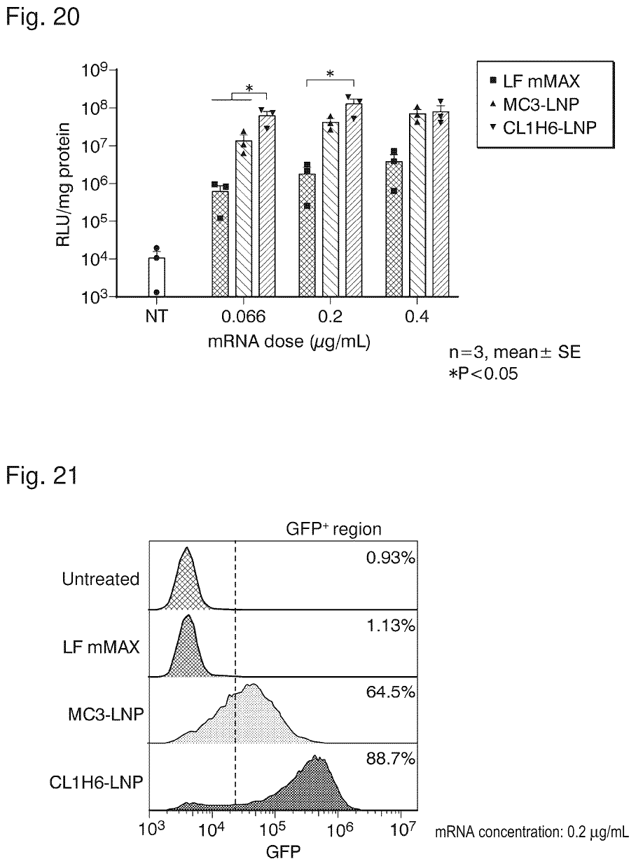

NK-92MI cells were transfected with each Luc mRNA-encapsulated lipid nanoparticle or the Lipofectamine-mRNA complex, and luciferase activity 24 hours later was examined by the method described in the section <Evaluation of ability to deliver Luc mRNA>. The measurement results are shown in FIG. 20.

As shown in FIG. 20, CL1H6-LNP exhibited higher luciferase activity than that of MC3-LNP.

### Example 8

GFP mRNA-encapsulated lipid nanoparticles were prepared using CL1H6 and Dlin-MC3.

(Preparation of CL1H6-LNP)

CL1H6-LNP was prepared in the same manner as the method for preparing CL1H6-LNP in Example 6 except that GFP mRNA was used instead of Luc mRNA. The physical properties of the lipid nanoparticle (n=3, mean±SE) are shown in Table 7.

(Preparation of MC3-LNP) MC3-LNP was prepared in the same manner as the method for preparing MC3-LNP in Example 6 except that GFP mRNA was used instead of Luc mRNA. The physical properties of the lipid nanoparticle (n=3, mean±SE) are shown in Table 7.

TABLE 7

| GFP | | | | Encapsu- | |
|---|---|---|---|---|---|
| mRNA- | Particle | | Zeta | lation | Recovery |
| encapsulated | size | | potential | rate | rate |
| LNP | (nm) | PDI | (mV) | (%) | (%) |
| CL1H6-LNP | 122 ± 6 | 0.152 ± 0.036 | 9.2 ± 0.4 | 70.7 ± 1.1 | 74.1 ± 4.3 |
| MC3-LNP | 117 ± 3 | 0.090 ± 0.029 | −3.8 ± 0.3 | 79.6 ± 0.2 | 85.0 ± 2.1 |

(Preparation of Lipofectamine-mRNA complex)

A Lipofectamine-mRNA complex was prepared by the method described in the section <Preparation of Lipofectamine-mRNA complex>using GFP mRNA as mRNA.

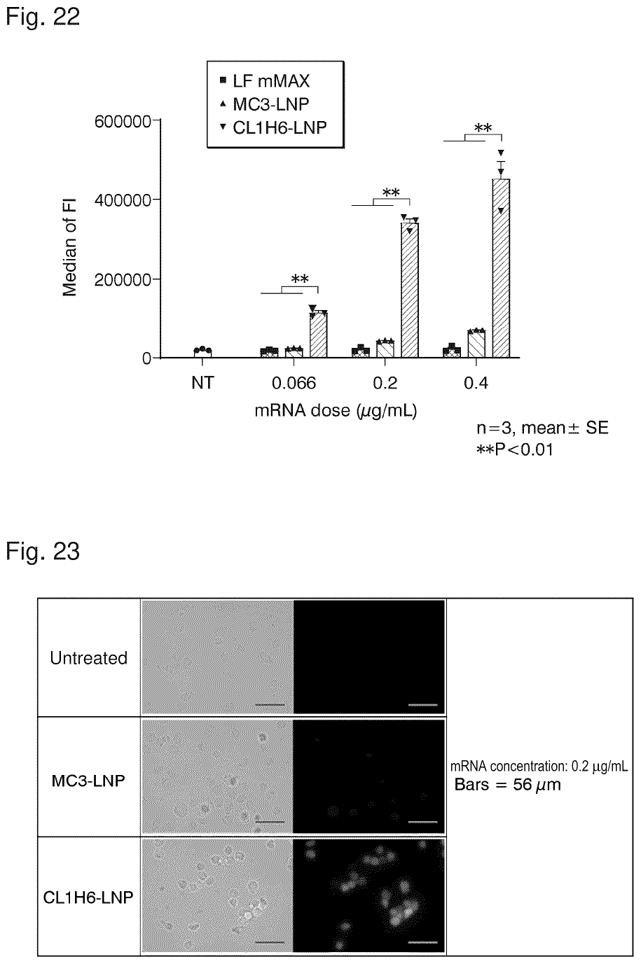

NK-92MI cells were transfected with each GFP mRNA-encapsulated lipid nanoparticle or the Lipofectamine-mRNA complex, and GFP expression 24 hours later was examined by the methods described in the sections <Evaluation of ability to deliver GFP mRNA> and <GFP observation under fluorescence microscope>. The measurement results are shown in FIG. 21 (histogram), FIG. 22 (median value of fluorescence intensity (FI)), and FIG. 23 (fluorescence microphotograph).

As shown in FIGS. 21, 22, and 23, CL1H6-LNP exhibited markedly higher GFP expression than that of MC3-LNP.

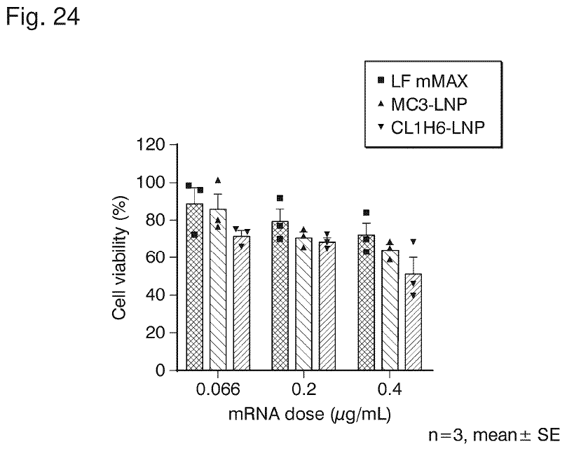

Subsequently, NK-92MI cells were transfected with each GFP mRNA-encapsulated lipid nanoparticle or the Lipofectamine-mRNA complex, and cytotoxicity 24 hours later was examined by the method described in the section <Evaluation of toxicity of mRNA-encapsulated lipid nanoparticle>. The measurement results are shown in FIG. 24.

As shown in FIG. 24, no cytotoxicity was exhibited at the concentrations that sufficiently produced GFP expression.

1. A lipid nanoparticle comprising a pH-sensitive cationic lipid represented by the following formula (1): 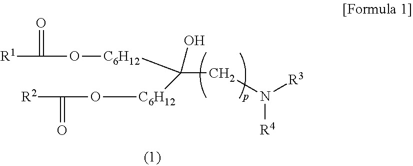
wherein R¹ and R² are each independently a straight-chain C₁₀₋₁₄ alkyl group, a straight-chain C₁₀₋₂₀ alkenyl group having one or two unsaturated bonds, or —CH(R⁵)(R⁶), where R⁵ and R⁶ are each independently a straight-chain C₅₋₁₀ alkyl group; p represents an integer of 3-8; and R³ and R⁴ are each independently a methyl group or an ethyl group.

2. The lipid nanoparticle according to claim 1, further comprising sterol and a polyalkylene glycol-modified lipid.

3. The lipid nanoparticle according to claim 1, wherein a ratio of an amount of the pH-sensitive cationic lipid to the total amount of the lipids constituting the lipid nanoparticle is 20 mol % or more.

4. The lipid nanoparticle according to claim 1, comprising a nucleic acid.

5. The lipid nanoparticle according to claim 4, wherein the nucleic acid is siRNA or mRNA.

6. The lipid nanoparticle according to claim 4, wherein the nucleic acid is plasmid DNA.

7. The lipid nanoparticle according to claim 4, wherein the nucleic acid is a gene to be expressed in an NK cell, or a functional nucleic acid that controls gene expression in an NK cell.

8. An NK cell transfected with the lipid nanoparticle according to claim 1.

9. A pharmaceutical composition comprising the lipid nanoparticle according to claim 1.

10. The pharmaceutical composition according to claim 9 for use in gene therapy.

11. The pharmaceutical composition according to claim 9 for use in cancer treatment.

12. The pharmaceutical composition according to claim 11 for use in cancer immunotherapy.

13. A kit for transforming an NK cell, comprising the lipid nanoparticle according to claim 1.

14. A method for transforming an NK cell, comprising introducing the lipid nanoparticle according to claim 7 to the NK cell so that the NK cell is transformed with the nucleic acid contained in the lipid nanoparticle.

15. A method for suppressing a cancer, comprising administering a transformed NK cell obtained by the method for transforming an NK cell according to claim 14 to an animal having a cancer tissue to reduce a size of the cancer tissue or to suppress increase in size of the cancer tissue.

16. A method for expressing a gene or a functional nucleic acid, comprising administering the lipid nanoparticle according to claim 7 to a test animal so that the gene or the functional nucleic acid contained in the lipid nanoparticle is expressed in an NK cell of the test animal.

17. A pharmaceutical composition comprising the NK cell according to claim 8 as an active ingredient.
